(19) United States
(12) Patent Application Publication
(10) Pub. No.: US 2024/0350401 A1
(43) Pub. Date: Oct. 24, 2024

(54) TOPICAL DELIVERY SYSTEMS FOR ACTIVE COMPOUNDS

(71) Applicants: Yissum Research Development Company of the Hebrew University of Jerusalem Ltd., Jerusalem (IL); LYOTROPIC DELIVERY SYSTEMS LTD, JERUSALEM (IL)

(72) Inventors: Nissim GARTI, Ramat HaSharon (IL); Sharon GARTI LEVI, Modi'in (IL)

(73) Assignees: Yissum Research Development Company of the Hebrew University of Jerusalem Ltd., Jerusalem (IL); LYOTROPIC DELIVERY SYSTEMS LTD, Jerusalem (IL)

(21) Appl. No.: 18/756,671

(22) Filed: Jun. 27, 2024

Related U.S. Application Data
(63) Continuation of application No. 16/491,972, filed on Sep. 6, 2019.
(60) Provisional application No. 62/467,863, filed on Mar. 7, 2017.

(51) Int. Cl. A61K 9/00; A61K 9/06; A61K 31/136; A61K 31/723; A61K 47/10; A61K 47/12; A61K 47/44

(52) U.S. Cl. CPC A61K 9/0014; A61K 9/06; A61K 31/136; A61K 31/723; A61K 47/10; A61K 47/12; A61K 47/44

(57) ABSTRACT

The disclosure concerns viscous or gelled delivery systems based on oily nano-domains dispersed in a viscosified/gelled continuous aqueous phase, and suitable for prolonged and/or sustained topical delivery of various active compounds.

### TECHNOLOGICAL FIELD

The present invention concerns novel viscous or gelled delivery systems based on oily nano-domains dispersed in a viscosified/gelled continuous aqueous phase, and suitable for prolonged and/or sustained topical delivery of various active compounds.

### BACKGROUND OF THE INVENTION

Topical delivery systems of active agents are often based on lipophilic carriers, that solubilize the active agent therein, such as ointments based on petroleum jelly, liquid paraffin or other oily carriers. Other delivery systems are emulsion-based creams and ointments, in which droplets of oil, into which the active agent is dissolved, are dispersed in an aqueous phase. Although various commercial products for topical delivery of actives exist, topical delivery of active agents from such systems have proven to be challenging from the formulatory aspect, the delivery profile and performance.

In particular, formulating such systems into topical formulations which combine long-term stability, a desired release profile of the active, controlled penetration into the skin layers (i.e. tailored to have limited systemic effect or prevent such effect), as well as being texturally satisfactory, has been difficult to obtain.

Thus, the present disclosure provides sub-micronic structures, i.e. nano-domains delivery systems, which are self-assembled, that are based on a unique multi-components oily phase that has low content of oil, and is dispersed in a viscosified or gelled continuous aqueous phase. Such systems are designed to load various active agents, and are suitable for topical administration of the active in a controlled, typically prolonged, release manner. Further, as will be described herein, the viscous/gelled formulations enable to obtain a depot effect within a desired skin layer to enable increase delivery of the active material, as well as prolonged and substantially constant release reate of the active upon administration. These systems, although composed of several components, are isotropic, self-assembled systems (i.e. formed spontaneously), thermodynamically stable, present high solubilization capacity, and have improved bioavailability of the active agent. Other advantages of these systems will become apparent from the disclosure below.

### REFERENCES

[1] WO 2008/058366
[2] A. Kogan, N, Garti, Advances in Colloid and Interface Science 2006, 123-126, 369-385
[3] A. Spernath, A. Aserin, Advances in Colloid and Interface Science 2006, 128
[4] A. Spernath, A. Aserin, N. Garti, Journal of Colloid and Interface Science 2006, 299, 900-909
[5] A. Spernath, A. Aserin, N. Garti, Journal of Thermal Analysis and Calorimetry 2006, 83
[6] N. Garti, A. Spernath, A. Aserin, R. Lutz, Soft Matter 2005, 1
[7] A. Spernath, A. Aserin, L. Ziserman, D. Danino, N. Garti, Journal of Controlled Release 2007, 119
[8] WO 03/105607
[9] J. Lademann, U. Jacobi, C. Surber, H. J. Weigmann, J. W. Fluhr, European Journal of Pharmaceutics and Biopharmaceutics 2009, 72, 317-323
[10] WO 2016/038553
[11] WO 2010/045415
[12] WO 2007/065281
[13] WO 1997/042944
[14] WO 1993/000873
[15] WO 2008/065451 A2
[16] WO2005/110370
[17] WO2007/060177

### SUMMARY OF THE INVENTION

The present disclosure concerns topical formulations for dermal (i.e. topical) delivery of an active agent, that provide a prolonged and enhanced release of the active agent by forming a depot effect at a desired skin layer. The unique combination of components in the topical formulation enables to obtain high penetration through the Stratum Corneum (however may be tailored for controlled penetration to limit or avoid systemic effects), while obtaining a controlled desired release profile of the active over a prolonged period of time, as described herein. While existing viscosified/gelled preparations that are emulsions or dispersions have proven to have limited thermodynamic stability and/or provide limited penetration of the active agent (see, for example, LUMiFuge™ test results of commercial emulsions shown in FIG. 1), formulations of the present disclosure demonstrate high stability over prolonged periods of time, high levels of penetration of the active agent carried therein, controlled and prolonged release of the active agent, as well as improved sensorial properties. It is also noted that formulations of the present disclosure are tailored to provide full dissolution of the active agent within the formulation, thus ensuring long term stability of the formulation and reproducible release of the active agent from the formulation upon application onto the skin.

In one of its aspects, the present disclosure provides a topical formulation comprising an oily phase and a gelled aqueous continuous phase, the oily phase being in the form of oily domains droplets that are dispersed in the gelled aqueous continuous phase; wherein the oily phase comprises an active agent or a pharmaceutically acceptable salt thereof, at least one oil, at least two hydrophilic surfactants, at least one co-surfactant (e.g. a lipophilic co-surfactant), at least two polar solvents, and at least two penetrating promotors, and the gelled aqueous continuous phase comprises an aqueous diluent and at least one gellant.

In another aspects, the present disclosure provides a topical formulation comprising an oily phase and a gelled aqueous continuous phase, the oily phase being in the form of oily nano-domains that are dispersed in the gelled aqueous continuous phase; wherein the oily phase comprises an active agent or a pharmaceutically acceptable salt thereof, at least one oil, at least two hydrophilic surfactants, at least two polar solvents, and at least two penetrating promotors and optionally comprising at least one co-surfactant (e.g. a lipophilic co-surfactant), and the gelled aqueous continuous phase comprises an aqueous diluent and at least one gellant.

The topical formulations comprise active-loaded delivery system, that are constituted by an oily phase in the form of distinct domains (e.g. droplets, that may or may not be spherical) that are dispersed in a continuous aqueous phase. The continuous phase is a gel, such that the formulation is viscosified to a consistency that allows obtaining a long residence period onto the skin once applied, as well as a pleasant and smooth texture. As noted above, the formulations are self-assembled systems (i.e. formed spontaneously), and tailored to solubilize and stabilize the active agent on the one hand, while permitting high skin penetrability and prolonged release of the active from the formulation once delivered into a desired skin layer on the other hand. Unlike typical emulsion or microemulsion formulations, these self-assembled structures are poor in oily phase, and as will be further explained below also contain a very low content of oil in the oily phase. The oily phase is constituted by nano-clusters or short domains of oil and surfactants, cosolvents and cosurfactants, however differ from the classical reverse micelles or reverse swollen micelles. When mixed with more than 60 wt % of water, oily domains structured from the surfactants and the active agent itself are formed; namely in the oily domains of the formulation, the active agent functions as a surface active agent (“structurant” or “cosmotropic agent”) located at the interface of the oily phase or being incorporated into the interface, being a part of the structure of the domain and enabling the formation of the oily domains. The unique oily phase used in the formulations of this disclosure, thus, differs from known topical delivery system in which the active agent is a mere guest molecule, i.e. typically solubilized into oil or an oily phase without significantly influencing the structure of the formulation. By tailoring the oily phase to enabling entrapment of the active agent between the surfactants' tails, the active agent is incorporated into the structure of the oily domain and functions to stabilize the domains' structure. In other words, in formulations of this disclosure, the active agent is solubilized within the interface of the oily/surfactant domains, thus forming a structural part of the oily domains and the interface rather than merely being solubilized in the core of the oily domain.

The formulations of the invention are thermodynamically stable submicronic-structures (having submicronic-size domains), which may be safely stored for prolonged periods of time, without creaming, aggregation, coalescence or phase separation, and are characterized by a substantially uniform and stable oily nano-sized domains, typically having a narrow size distribution within the aqueous phase. In addition to formulation stability considerations, the uniformity of domains' size and their size distribution permits better control of the active's rate of release from the formulation as well as enhanced transport/permeation into the skin.

It should be emphasized that the structure of formulations of this disclosure are formed spontaneously once the active agent is introduced into the oily mixture and the aqueous component is added at a required amount (i.e. above ca. 60%), without the need to apply high shear, cavitation or high-pressure homogenization processes, but rather upon simple mixing of the components at low mixing rates. In some embodiments, the oily domains (in the gelled system) have a size of between about 5 and 150 nm (nanometers), or even between 10 and 100 nm. The domain size refers to the arithmetic mean of measured domain's diameters, wherein the diameters range ±15% from the mean value.

In other embodiments, the oily domains (in the gelled system) may have a size of between about 10 and 75 nm, between about 10 and 50 nm, or even between 10 and 25 nm. In some other embodiments, the oily domains (in the gelled system) may have a size of between about 15 and 75 nm or even between about 20 and 50 nm.

It is of note that the domains need not be spherical. In some embodiments, the oily domains in the formulation have an elongated shape, namely, having an ellipsoid, oblong or worm-like shape with at least 2 different dimensions. In such cases, the average domain size refers to the imaginary sphere having a diameter of the longest dimension of the domain.

In some embodiments, the elongated oily domains have an aspect ratio of between about 1.1 and 1.5.

Control of skin permeability and rate of release is also obtained by tailoring the formulations' viscosity, i.e. by jellifying the aqueous phase to form slight to medium viscosity formulation, typically in the form of a gel. It was found by the inventors of the present invention that the delivery system can be viscosified to a desired viscosity with controlled rheological properties, thereby increasing the stability of the system, and prolonging the release of an active from the formulation once topically administered. The controlled increase of viscosity also permits improving the spreadability of the formulation onto the skin, as well as providing longer contact time between the skin and the formulation, as will be explained herein.

It should be noted that the formulations of the present disclosure are capable of maintaining their nano-size without interacting with the gel molecules and without being flocculated or coalesced, and remain mobile within the gelled phases. This unique characteristic is achieved by selection of gellants that have no surface activity and that do not interact with the active agent.

In the context of the present disclosure, the term viscous or any lingual variation thereof, both when referring to the aqueous phase and/or the formulation, means to denote a viscosity larger than that of water (i.e. viscosity higher than 1 cP at 25° C.). Typically, the gelled aqueous phase has a viscosity of at least 100 cP (centipoise or mPa/s) on its own, while the formulation may have a viscosity of at least 400 cP (measured by Brookfield DV-II viscometer, 15 rpm with Spindle LV4, at a gellant concentration of 2.85 wt %). Unless specifically indicated, all viscosity values described herein refer to viscosity as measured at 25° C.

The increased viscosity is obtained predominantly by the use of a gellant, specifically a gellant that does not affect the structure of the nano-domains, to be described further herein. The gellant forms a three-dimensional molecular network in the aqueous phase, thereby increasing the formulation's viscosity. It is also of note that, since the nano-domain structures in the gel phase are not Newtonian, the rheological behavior may be an indicator of the viscosity behavior of the system. The empty (i.e. without the active) and the loaded nano systems have higher loss modulus (G′) and storage modulus (G″) than those of the aqueous gel (i.e. a gelled aqueous phase without the nano-domains), showing similar rheological behavior to soft viscoelastic gels.

The complex viscosity of the gelled nano-domains is higher than that of the aqueous gelled phase at low shear rate (12 vs. 8 Pa·s at 0.1 1/s shear rate), but is equal to the gelled system at higher shear rates of ca. 0.6 Pa·s at 80 1/s.

The viscosity of the gelled formulation does not change as a function of storage time and is fully reproducible even after few shear stress cycles, indicating that the nanodomains are not attached to the viscoelastic network. In addition, contrary to formulations that need to be fully absorbed into the skin, the gelled formulation forms a thin film on the surface of the skin once applied. This film has a longer residence time on the skin, thereby increasing the contact time of the formulation with the skin, that enables the active agent to diffuse out of the oily domains and into the skin deeper layer (causing a depot effect) during a longer period of time.

Topical formulation refers herein to a formulation adapted for dermal application and enables dermal and/or transdermal delivery of the active agent. The term as used herein refers to the application of a formulation directly onto at least a portion of a subject's skin (human's or non-human's skin) so as to achieve a desired effect, e.g. cosmetic or therapeutic effect, at the site of application and neighboring area or tissues. In some embodiments, the desired effect is achieved at the site of application without inducing one or more systemic effects. In other embodiments, the formulation of this disclosure induces at least a partial, limited, systemic effect which contributes to the induction of at least one desired effect.

As known, human skin is made of numerous layers which may be divided into three main group layers: Stratum Corneum which is located on the outer surface of the skin, the epidermis and the dermis. While the Stratum Corneum is a keratin-filled layer of cells in an extracellular lipid-rich matrix, which in fact is the main barrier to drug delivery into skin, the epidermis and the dermis layers are viable tissues. The epidermis is free from blood vessels, but the dermis contains capillary loops that can channel therapeutics for transepithelial systemic distribution.

While dermal delivery of drugs may be a route of choice, only a limited number of drugs can be administered through this route. The inability to dermally deliver a greater variety of drugs depends mostly on the requirement for low molecular weight (drugs of molecular weights not higher than 500 Da) to facilitate skin penetration, lipophilicity and relatively small doses of the drug that may be loaded into known carriers. The formulations of this disclosure permit the transport of the active agents across at least one of the skin layers, across the Stratum Corneum (SC), the epidermis and the dermis layers. Without wishing to be bound by theory, the ability of the delivery system to transport the active agent across the Stratum Corneum depends on a series of events that include controlled diffusion of the active agent through a hydrated keratin layer and into the deeper skin layers. Such controlled diffusion is enabled by the combination of the increased viscosity together with the interface interactions of the active agent and the surfactants of the oily phase, as will be further explained.

In some embodiments, the formulations are adapted for epidermal and/or dermal administration of at least one active agent. In other embodiments, the formulation may be adapted for delivery of the active agent across skin layers, and specifically across the Stratum Corneum. In some other embodiments, the formulation is adapted for dermal delivery of the active agent without causing significant systemic effect. In yet other embodiments, the formulation is adapted to deliver the active agent through the Stratum Corneum to induce an effect at a desired tissue (muscle, synovial fluid, synovial membrane, patellar tendon, etc.).

Within the scope of this disclosure, the term skin refers to any region of a mammalian skin (including human skin), including skin of the scalp, hair and nails. The skin region to which the formulation may be applied, depends inter alia on parameters discussed herein.

In another aspect, there is provided a topical formulation for providing prolonged release of at least one active agent, the formulation comprising an oily phase and a gelled aqueous continuous phase, the oily phase being in the form of oily domains that are dispersed in the gelled aqueous continuous phase; the oily phase comprises said active agent, at least one oil, at least two hydrophilic surfactants, at least one co-surfactant, at least two polar solvents, and at least two penetrating promotors, and the gelled aqueous continuous phase comprises an aqueous diluent and at least one gellant; said the formulation being adapted to form a film onto a skin region once applied thereonto such that said active agent being released from said oily droplets for a period of time upon contact with the skin region, thus providing prolonged and increase release thereof.

In another aspect, there is provided a topical formulation for providing prolonged release of at least one active agent, the formulation comprising an oily phase and a gelled aqueous continuous phase, the oily phase being in the form of oily domains that are dispersed in the gelled aqueous continuous phase; the oily phase comprises said active agent, at least one oil, at least two hydrophilic surfactants, at least one co-surfactant, at least two polar solvents, and at least two penetrating promotors, and the gelled aqueous continuous phase comprises an aqueous diluent and at least one gellant; said active agent being only physically associated with the oily domains and the aqueous phase, permitting the active agent to be released from said oily domains upon contact with a skin region for a prolonged and increased period of time.

The formulations described herein may provide prolonged release of the active agent once topically administered; namely, the active agent is released from the formulation into the desired administration site over a period of time of at least 12 hours from administration. In some embodiments, the active agent is released from the formulation over a period of time of at least 24 hours, at times up to 48 hours, once applied onto the skin of a subject. In some embodiments, when measured by Franz cell measurements, the accumulated amount of permeated active agent is increased by about 2-folds every 2 hours during a period of 0.5-12 hours from application, and/or about 6-folds over a period of 12 to 24 hours, and by 2-folds from 24 to 48 hours from application.

It is of note that when measured by a Franz cell, the amount of active agent within the receiver vessel (mimicking the blood stream) is minimal, e.g. between 0.5 to 2% from the total applied active agent, showing minimal systemic exposure.

In other embodiments, the accumulated amount of the active agent in the surface skin layers over 24 hours from application is at least 4-8% from the amount applied onto the skin.

Formulations of this disclosure may also provide a depot effect, in which, once administered to a desired skin layer, the formulation functions as a reservoir of the active agent, from which the active agent is released in a controlled manner over a defined period of time. Namely, the formulations of this disclosure are designed to form a thin film of gelled formulation onto a skin area once applied thereonto. Due to the unique structure of the oily domains and careful tailoring of diffusion coefficients of components in the formulation, the active agent is being released in a controlled manner (constant rate) over a prolonged period of time from the oily phase into the skin, once the formulation comes into contact with the skin (as will be explained in a detailed manner herein).

Thus, in a further aspect, there is provided a depot formulation for topical delivery of at least one active agent, the formulation comprising an oily phase and a gelled aqueous continuous phase, the oily phase being in the form of oily domains that are dispersed in the gelled aqueous continuous phase; wherein the oily phase comprises said active agent, at least one oil, at least two hydrophilic surfactants, at least one co-surfactant, at least two polar solvents, and at least two penetrating promotors, and the gelled aqueous continuous phase comprises an aqueous diluent and at least one gellant; said active agent having a diffusion coefficient in the formulation similar to that of the hydrophilic surfactant, and said aqueous phased is gelled, permitting the active agent to be released from said oily domains upon contact with a skin region over a prolonged period of time.

The term obstruction factor (OF) is defined as the diffusivity (diffusion coefficient) of each component in the formulation normalized to diffusion coefficient of the component itself in a liquid form or in a reference solution [OF=D/D₀]. The obstruction factor is suggestive of the resistance of the components in the oily domains to be released from the structure at a given concentration of the active agent. Low OF values are indicative to binding effects between components having similar OF values.

As noted above, formulations of this disclosure are constituted by oily domains dispersed in the gelled aqueous continuous phase. The oily phase is a unique multi-component mixture that is substantially (at times entirely) devoid of water, and comprises at least one oil, at least two hydrophilic surfactants, at least one co-surfactant (typically a lipophilic co-surfactant), at least two polar solvents, and at least two penetrating promotors. Contrary to classic emulsion or microemulsion systems, which are rich in oil so that the active agent is typically dissolved within an oil core, in the formulations of this disclosure the oily phase is poor in oil. This low content of oil is insufficient for solubilizing the active agent, thus forcing the active agent to be entrapped within the tails of the surfactants, and hence reside at the interface between the oily domains and the aqueous phase. Such solubilization within the interface of the oil-surfactant results in highly thermodynamically stable formulation, that does not undergo phase separation or release of the active agent from the droplet over prolonged period of time, while upon contacting a biological membrane the active can be released from the formulation. Due to the combination of the oily phase components, the oily phase may be loaded with relatively high contents of the active agent, e.g. up to 20 wt % or more, typically up to 15 wt % of the oily phase (it is of note, however, that once diluted with an aqueous carrier, the concentration of the active agent within the entire formulation should be recalculated according to the relevant degree of dilution).

Once the aqueous phase is added to the oily phase, the oily phase rearranges to form oily domains. Due to the careful tailoring of components, the system is spontaneously arranged into its final structure, driven by the structural match between the surfactants, co-surfactant and the active agent (i.e. high molecular compatibility), as well as the formation of an interface having a substantially zero interface tension. Such matching of components provides for a system exhibiting interface elasticity that enables the curvature of the interface that spontaneously forms between the oily domains and the aqueous phase to modify in order to accommodate the active agent and facilitate its physical interactions with the surfactants tails. The system is also tailored to enable an effective critical packing factor (ECPP) at the interface, as well as suitable obstruction factor (as will be explained herein), thus stabilizing the oily domains on the one hand and enabling enhanced release of the active from the domain once coming to contact with the skin on the other hand.

Thus, in another aspect, the disclosure provides a viscous topical formulation comprising an oily phase in the form of oily domains that are dispersed in a gelled aqueous continuous phase, wherein the oily phase comprises at least the following 9 components: said active agent, at least one oil, at least two hydrophilic surfactants, at least one lipophilic co-surfactant, at least two polar solvents, and at least two penetrating promotors, and the gelled aqueous continuous phase comprises an aqueous diluent and at least one gellant.

The oil refers to a lipophilic agent which is immiscible in water and is capable of forming distinct domains when introduced into an aqueous liquid. In some embodiments, the oil is selected from isopropyl-myristate (IPM), ethyl oleate, methyl oleate, lauryl lactate, oleyl lactate, oleic acid, linoleic acid, monoglyceride oleate and monoglyceride linoleate, coco caprylocaprate, hexyl laurate, oleyl amine, oleyl alcohol, hexane, heptanes, nonane, decane, dodecane, short chain paraffinic compounds, terpenes, D-limonene, L-limonene, DL-limonene, olive oil, soybean oil, canola oil, cotton oil, palmolein, sunflower oil, corn oil, essential oils, such as peppermint oil, pine oil, tangerine oil, lemon oil, lime oil, orange oil, citrus oil, neem oil, lavender oil, anise oil, pomegranate seed oil, grape seed oils, rose oil, clove oil, sage oil, eucalyptol oil, jasmine oil, oregano oil, capsaicin and similar essential oils, triglycerides (e.g. unsaturated and polyunsaturated tocopherols), medium-chain triglycerides (MCT), avocado oil, grapeseed oils, pumpkin oil, punicic (omega 5 fatty acids) and CLA fatty acids, omega 3-, 6-, 9-fatty acids and ethylesters of omega fatty acids and mixtures thereof.

In other embodiments, the oil may be selected from isopropyl-myristate (IPM), oleic acid, oleyl alcohol, vegetable oils, terpenes, peppermint oil, eucalyptol oil, and mixtures thereof.

In another embodiment, the oil is isopropyl-myristate (IPM).

As noted above, the oily phase is poor in oil, in order to drive the active agent towards the interface, rather than causing solubilization of the active within the oil. Thus, according to some embodiments, the oil may be present in the formulation in an amount of at most 3 wt %. According to other embodiments, the oil may be present in the formulation at an amount of between about 0.5 and 3 wt % from the formulation. According to some other embodiments, the oil may be present in the formulation at an amount of about 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or even 3 wt % from the formulation. According to yet other embodiments, the oil may be present in the formulation in an amount of between about 0.5 and 1 wt % from the formulation.

The hydrophilic surfactants are surface-active agents that have a hydrophilic head group and lipophilic tails that are capable of solubilizing the active agent. The head groups are capable of interacting with the active agent and the penetrating agents, thus allowing formation of the oily domains. Depending on the active agent to be loaded into the formulation, the hydrophilic surfactants may include, ionic, cationic zwiterionic or non-ionic surfactants having a hydrophilic nature (i.e. having large head groups), thereby providing a surfactant having an affinity for water. Exemplary surfactants are polyoxyethylene sorbitan monolaurate (polysorbate 20 or T20), polyoxyethylene sorbitan monopalmitate (T40), polyoxyethylene sorbitan monooleate (T80), polyoxyethylene sorbitan monostearate (T60) and polyoxyethylene esters of saturated (hydrogenated) and unsaturated castor oil (such as HECO25, HEC040, HECO60, EC035, EC040, EC060, PEG 25, PEG40, PEG45, PEG60 ethylene glycols, PEG45 palm kernel and others, ethoxylated monoglycerol esters (such as PEG 5, 6, 7, 20, 40—caprylic/capric, lauric and oleic glycerides), hydroxystearate, ethoxylated fatty acids and ethoxylated fatty alcohols of short and medium and long chain fatty acids, sugar esters of saturated and unsaturated fatty acids, mono- and polyesters of sucrose, polyglycerol esters (3, 6, 8, 10 glycerols) of fatty acids, ethoxylated mono glycerides (8, 10, 12, 20, 40 EO) and ethoxylated diglycerides, ethoxylated fatty acids and ethoxylated fatty alcohols.

The oily phase comprises at least two hydrophilic surfactants. The hydrophilic surfactants are selected and matched such that their combination forms a “Sherman complex”. The Sherman complex refers to a set of two or more surfactants that form dense, well-packed and compacted interfacial layer, resulting from a match of the two surfactants with two lipophilic tails; namely, one having a longer tail and the other having a shorter tail that are integrated one into the other in the core of the domain. In the Sherman complex, the two surfactants have hydrophilic head groups, the first with larger head group and the other with a smaller head group, forming strong hydrogen bonding between the head groups. Such complexes provide increased solubilization of the bioactives (active agent) in the nano-domains and better chemical stabilization of the active agent within the tails of the surfactants.

In other words, the formulation comprises a first hydrophilic surfactant and a second hydrophilic surfactant, provided that the first hydrophilic surfactant is different from the second hydrophilic surfactant.

In some embodiments, each of the hydrophilic surfactants may be selected from polyoxyethylenes, ethoxylated (20EO) sorbitan monolaurate (T20), ethoxylated (20EO) sorbitan monostearate/palmitate (T60), ethoxylated (20EO) sorbitan mono oleate/linoleate (T80), ethoxylated (20EO) sorbitan trioleate (T85), castor oil ethoxylated (20EO to 60EO); hydrogenated castor oil ethoxylated (20 to 60EO), ethoxylated (5-40 EO) monoglyceride stearate/palmitate, polyoxyl 35 and 40 EOs castor oil. According to other embodiments, each of the hydrophilic surfactants may be independently selected from polyoxyl 35 castor oil, polysorbate 20 (Tween 20), polysorbate 40 (Tween 40), polysorbate 60 (Tween 60), polysorbate 80 (Tween 80), Mirj S40, Mirj S20, oleoyl macrogolglycerides, Polyglyceryl-3 dioleate, ethoxylated hydroxyl stearic acid (Solutol HS15), sugar esters such as sucrose mono oleate, sucrose mono laurate, sucrose mono stearate, Polyglycerol esters such as deca glycerol mono oleate or monolaurate, hexa glycerol monolaurate or mono oleate.

In some embodiments, the first hydrophilic surfactant may be selected from polysorbate 40 (T40), polysorbate 60 (T60), polysorbate 80 (T80), Mirj S40, oleoyl macrogolglycerides, polyglyceryl-3 dioleate, ethoxylated hydroxyl stearic acid (Solutol HS15), or sugar esters, while the second surfactant may be selected from castor oil ethoxylated (20EO to 40EO); hydrogenated castor oil ethoxylated (20 to 40EO).

According to some embodiment, the first hydrophilic surfactant is polysorbate 60 (Tween 60) and the second hydrophilic surfactant is hydrogenated castor oil (40EO, HECO 40).

In some embodiments the ratio between the first and second hydrophilic surfactants is between about 5:1 and 2:1 (w/w).

In some embodiments, the first hydrophilic surfactant may be present in the formulation in an amount of between about 1.75 and 8.0 wt %, while the second hydrophilic surfactants may be present in an amount of between about 0.45 and 3.8 wt %. In other embodiments, the first hydrophilic surfactant may be present in the formulation in an amount of between about 2.5 and 4.5 wt %, while the second hydrophilic surfactants may be present in an amount of between about 0.5 and 1.8 wt %. According to some other embodiments, the total amount of hydrophilic surfactants in the formulation is between 2 and 12 wt %.

The formulation comprises at least two polar solvents. In the context of the present disclosure, the term solvent refers to any polar organic solvent that is water miscible and is suitable for assisting the solubilization of the active agent. The combination of two polar solvents is used to facilitate full coverage of the interface by the hydrophilic surfactant at any water dilution of the formulation. The solvents provide the solubilization conditions for the progressive migration of the surfactants to the interface upon dilution. At low water contents the solvents are essential components to render the behavior of the hydrophilic surfactant to be lipophilic-like, adjusting its effective critical packing parameter (ECPP) to >1.3, and at high water levels (>50%) the solvents are “pushing” the surfactants to the interface and causing a significant alternation of the ECPP to <0.5. In other words, the hydrophilic surfactants are controlling and adjusting the hydrophilicity/lipophilicity of the surfactants at any water content. Thus, the combination of solvents is required to allow complete geometrical packing of the two solvents to fill up the space (voids) in between the surfactants at the interface.

Thus, in some embodiments, the formulation comprises at least a first solvent and a second solvent, provided that the first solvent being different from the second solvent.

According to some embodiments, the first polar solvent may be selected from short chain alcohols (e.g. ethanol, propanol, isopropanol, butanol, etc.), while the second polar solvent may be selected from polyols (e.g. propylene glycol (PG), glycerol, xylitol and other monomeric or dimeric sugar units, and polyethylene glycol (PEG), such as PEG 200, PEG 400, etc.).

In some embodiments, the formulation may comprise isopropanol (IPA) as the first polar solvent, and propylene glycol (PG) as the second polar solvent. In other embodiments, the formulation may comprise ethanol as the first polar solvent, and propylene glycol as the second polar solvent.

According to other embodiments, the formulation comprises at least three solvents. In such embodiments, the formulation may comprise IPA, ethanol and PG as polar solvents.

According to further embodiments, the first polar solvent is selected from ethanol, IPA, and combinations thereof.

Without wishing to be bound by theory, the first polar solvent(s) is located deeper within the interface (i.e. ethanol and/or IPA). The first polar solvent(s) functions to provide the elasticity of the curvature (R^{e}) and spontaneous curvature (R^{s}, or R^{o}) of the oily domain interface with the aqueous phase. The second polar solvents (i.e. the polyol) is located close to the surfactant head groups, thereby dehydrating them and solubilizing the active agent.

In some embodiments, the ratio between the first solvent(s) and the second solvent is between about 1:1.5 and 1:3.

In some embodiments, total amount of solvents in the formulation is between about 2.5 and 25 wt %. In other embodiments, the total amount of the solvents in the formulation may be between about 3 and 20 wt %, between about 3.5 and 18 wt %, between about 4 and 16 wt %, or even between about 5 and 15 wt %.

As noted above, the active-surfactants-solvents system forms strong molecular interactions, thus permitting solubilization and stabilization of the active agent within the interface of the oily domains. The combination of the surfactants and active agent in the presence of the solvents provides for interactions between the surfactants and the active agent (i.e. physical binding of the active agent to the surfactant molecules), thereby inhibiting the active agent from migrating from the oily domain into the aqueous phase, thus increasing the formulation's shelf life.

Another component of the oily phase is at least one co-surfactant, typically a lipophilic or an amphiphilic co-surfactant, which in some embodiments, may be present in the formulation in an amount of between about 0.4 and 2.0 wt %. In other embodiments, the co-surfactant may be present in the formulation in an amount of between about 0.45 and 1.8 wt %, or even between about 0.5 and 1.5 wt %. The term co-surfactant should be understood to encompass any lipophilic or amphiphilic agent, different from the surfactants, which contributes (together with the surfactants) to lowering of the interfacial tension between the oily phase and the aqueous phase to almost zero (or zero) allowing for the formation of thermodynamically stable oily domains.

According to some embodiments, the co-surfactant may be a phospholipid.

The phospholipid forming a component of the oily phase is typically lipophilic or amphiphilic to induce a structural change or temporary disorder in a biological lipid membrane upon contact (fusion); the structural change may be one or more of alteration of membrane curvature, modification of surface charge, promotion of nonbilayer lipid phases, adhering to the membrane, and altered phospholipid headgroup spacing within the bilayer.

In some embodiments, the phospholipid may be a glycerophospholipid being selected from mono-phosphatidyl glycerols, bis-phosphatidyl glycerols, and tris-phsophatidyl glycerols. Non-limiting examples of such phospholipids are phosphatidyl choline (PC), dipalmitoylphosphatidylcholine (DPPC), distearoyl phosphatidyl choline (DSPC), palmytoyl strearoyl phosphatidyl choline (DSDC), palmitoyl oleyl choline (PODC) and any other mixed fatty acids glycerolphosphatidyl choline, any phosphatidyl ethanolamine (PE) (Cephalin), any phosphatidyl inositol (PI), any phosphatidyl serine (PS), cardiolipin, plasmalogen, lyso phosphatidyl choline (LPC), lysophosphatidic acid, phosphatidylinositol (3,4)-bisphosphate, phosphatidylinositol (3,5)-bisphosphate, phosphatidylinositol (4,5)-bisphosphate, phosphatidylinositol 4-phosphate, phosphatidylinositol (3,4,5)-trisphosphate, phosphatidylinositol 3-phosphate, soy lecithin, rapeseed lecithin, corn or sunflower lecithins, egg lecithin, Epicorn 200, Epicorn 100, phospholipone 90G, LIPOID R-100 (Rapeseed), LIPOID H-100 (Sunflower), LIPOID-S100 (Soybean), LIPOID-575, Phosal 50PG, dioleyl phosphatidylcholine (DOPC), oleyl palmytoyl phosphatidylcholine (POPC), and their corresponding serines, ethanol amines, glycerol, and others.

In other embodiments, the co-surfactant may be selected from lecithins, egg lecithins, soybean lecithins, canola or sunflower lecithins, phospholipids such as phosphatidylcholine (PC) (GMO—Genetically Modified Organism, and non-GMO), Phosal, phospholipones, Epicorn 200, LIPOID H100, LIPOID R100, LIPOID S100, LIPOID S75, POPC, SOPC, PHOSPHOLIPON 90G or PHOSPHOLIPON 90H and others, as well as combinations thereof.

The phospholipid may, by some embodiments, be present in the formulation in an amount of between about 0.4 and 2.0 wt %.

Increased penetration of the formulation into the skin may be at least partially obtained by the use of penetrating promotors, which are compounds capable of changing the polarity of the Stratum Corneum, thereby improving the penetration of the formulation therethrough. Without wishing to be bound by theory, the penetrating promotors function to locally and temporarily distort the phospholipid structure of the phospholipid membrane at the Stratum Corneum, thereby increasing the mobility of the Stratum Corneum molecular components (both lipids and proteins), thus rendering the Stratum Corneum more permeable to the active agent, which is typically lipophilic.

In some embodiments, the total amount of penetrating promotors in the formulation is between about 2 and 10 wt %. In other embodiments, the total amount of penetrating promotors in the formulation may be between about 2.2 and 10 wt %, between about 2.5 and 8 wt %, or even between 2.5 and 6 wt %.

The formulation comprises at least two penetrating promotors, the combination of which controls the permeation of the active agent into the desired skin layer. Hence, by selecting specific combinations of penetrating promotors, the active agent can be delivered to the dermis or the epidermis with only slight systemic exposure. In some embodiments, the combination of said two or more penetrating agents provides a synergistic penetration and permeation effect of the active agent.

According to some embodiments, the penetrating promotors may be selected from sulfoxide derivatives such as dimethyl sulfoxide (DMSO), dimethyl isosorbide (DMI), isopropyl myristate (IPM), 2-(2-ethoxyethoxy)ethanol (transcutol), phosphatidylcholine (PC), ethanol, isopropyl alcohol (IPA), ethyl acetate, oleyl alcohol, oleic acid, oleyl esters, beta- cyclodextrines, urea and its derivatives such as dimethyl or diphenyl urea, glycerol and propyleneglycol (PG), pyrrolidone and derivatives, peppermint oil, or terpene and terpenoids (essential oils) oils, as well as combinations thereof. According to some embodiments, the formulation may comprise at least two penetrating promoters selected from DMI, PC, terpenes and transcutol.

According to other embodiments, the formulation may comprise at least two penetrating promoters selected from DMI, PC, and transcutol.

In some embodiments, the formulation may comprise (i) DMI and transcutol, (ii) DMI and PC, (iii) DMI and terpenes, (iv) PC and terpenes, (v) transcutol and terpenes, or (vi) PC and transcutol, as penetrating promotors.

According to other embodiments, the formulation may comprise three penetrating promotors, which in some embodiments are DMI, transcutol and terpenes.

In some other embodiments, the formulation may comprise three penetrating promotors, which in some embodiments are DMI, transcutol and PC.

As noted above, the active agent or a pharmaceutically acceptable salt, hydrate, derivative or analogue thereof, is solubilized within the interface of the oil phase (i.e. between the tails of the surfactants that form the oily nano-domains).

The term pharmaceutically acceptable salt(s), as used herein, means those organic salts of the active agent that are safe and effective for pharmaceutical use in mammals and that possess the desired biological activity. Pharmaceutically acceptable salts include salts of acidic groups present in compounds of the invention.

In some embodiments, the active agent may be selected from diclofenac, diclofenac sodium (DCF-Na), diclofenac potassium (DCF-K), DCF-ammonium, diclofenac diethylamine (DCF-DEA) and mixtures thereof, or any other pharmaceutically acceptable salt of diclofenac.

In some embodiments, the formulation comprises said active agent in an amount of between about 1 and 6 wt %. In other embodiments, the formulation comprises said active agent in an amount of between about 1.5 to 5 wt % or even between about 2 and 4.5 wt %.

The continuous phase of the formulation is a gelled, viscous aqueous phase in which the domains of the oily phase are dispersed. As noted above, the aqueous phase is viscosified/gelled by a gellant. The gellant is an agent that is capable of contributing to the elasticity and increasing the viscosity of the aqueous phase to a desired viscosity in addition to the formation of thin film when in contact with the skin layer, and hence to increase the viscosity of the formulation, as described herein.

Gellants are agents that are capable of forming a 3-dimensional network of macromolecules, for example a viscoelastic network of polymeric chains, in which the oily domains are embedded and homogenously dispersed, thereby increasing the viscosity and modifying the rheological behavior of the aqueous phase. For example, the gellant may be selected from water-soluble or colloidal water-soluble polymers (hydro-colloids), such as cellulose ethers (e.g. hydroxyethyl cellulose, methyl cellulose, hydroxypropylmethyl cellulose), polyvinylalcohol, polyquaternium-10, guar gum, hydroxypropyl guar gum, xanthan gum (such as Keltrals, Xanturals such as Xantural 11K, Xantural 180K, Xantural 75 (CP Kelco US) and others), gellans (Kelogels), Aloe vera gel, amla, carrageenan, oat flour, starch and modified starch (from corn rice or other plants), gelatin (from porcine or fish skin), ghatty gum, gum Arabic, inulin (from chicory), Konjac gum, locust bean gum (LBG), fenugreek, marshmallow root, pectin (high and low methoxy) and modified pectins, quinoa extract, red alga, solagum, tragacanth gum (TG) and any mixtures thereof.

In other embodiments, the gellant may be selected amongst acrylic acid/ethyl acrylate copolymers and the carboxyvinyl polymers under the trademark of Carbopol resins. Examples include Carbopol 934, Carbopol 940, Carbopol 950, Carbopol 980, Carbopol 951 and Carbopol 981. Carbopol 934 is a water-soluble polymer of acrylic acid crosslinked with about 1 of polyallyl ether of sucrose having an average of about 5.8 allyl groups for each sucrose molecule. Also suitable for use herein are hydrophobically-modified crosslinked polymers of acrylic acid having amphipathic properties available under the Trade Name Carbopol 1382, Carbopol 1342 and Pemulen TR-1. A combination of the polyalkenyl polyether cross-linked acrylic acid polymer and the hydrophobically modified crosslinked acrylic acid polymer may also be suitable.

Other gellants may be those that are cross-linkable by a suitable linker compound, as to form a 3-domensional interconnected network of molecules. Exemplary gellants of this type are crosslinked maleic anhydride-alkyl methylvinylethers, and copolymers, commercially available as Stabilizes QM (International Specialty Products (ISP)), Carbomer, crosslinked polymethacrylate copolymer.

According to some embodiments, the gellant may be selected from xanthan gum, gellan, sodium alginate, pectin, low and high methoxy pectins and carbomers.

According to other embodiments, the gellant is xanthan gum or gellan.

In some embodiments, the formulation comprises an amount of between about 0.75 and 3.5 wt % of said at least one gellant.

The aqueous diluent that is viscosified/gelled by the gellant may be any suitable aqueous liquid, such as water, purified water, distilled (DW), double distilled (DDW) and triple distilled water (TDW), deionized water, water for injection, saline, dextrose solution, or a buffer having a pH between 4 and 8.

In some embodiments, the formulation comprises between about 50 and about 90 wt % of the diluent, typically ca. 65-80 wt %.

As a man of the art may appreciate, the ratio between the formulations' various components may be tailored according to the nature of the active agent and its desired loading into the formulation, and may also be selected for endowing certain characteristics to the formulation (such as, desired domain size and electrical charge).

In some embodiments, the formulations may further comprise various additives, such as perfume (such as pine oil, lavender oil, peppermint oil, orange oil, lemon oil, eucalyptus oils and formulated fragrances, Eucaliptol stings, etc.), pH adjusting agents and buffers (such as citric acid, phosphoric acid, sodium hydroxide, monobasic sodium phosphate, strong ammonia, mono-, di- and trimethylamine, mono-, di- and triethanol amine, etc.) neutralizing agents, emollients, humectants, preservatives (such as benzalkonium chloride or parabens (C₁-C7-alkyl esters of 4-hydroxybenzoic acid, e.g. methyl 4-hydroxybenzoate), cetrimonium bromide, benzethonium chloride, alkltrimethylammonium bromide, EDTA, benzyl alcohol, cetyl alcohol, steryl alcohol, benzoic acid, sorbic acid, potassium sorbate thimerosal, imidurea, bronopol, chlorhexidine, chloroactamide, trichlorocaraban, propyl paraben, methyl paraben, phenyl mercuric acetate, chlorobutanol, phenoxyethanol and combination thereof and mixtures thereof) and antioxidant (such as butylated hydroxyanisole (BHA), butylated hydroxytoluene (BHT), ascorbyl palmitate, ascorbic acid, TBHQ, tocopherol, tocopherol acetate and combinations thereof).

In contrast to the milky white commercially-available emulsion-based topical viscous formations, the presently disclosed formulations are typically transparent (or substantially transparent) due to their mono-dispersed submicronic oily domain size (having a domain size of up to 100 nm) and high stability, maintaining their transparency for a prolonged period of time. The small domain size, which are less than one fourth of the average wavelength of visible light (0.560 micrometer), appear to the naked eye as a clear and homogenous formulation, lacking any observable clouding or areas of phase separation. This permits easy detection of changes in the formulation's stability (as phase separation, bioactive precipitation, and/or coalescence of oil droplets will cause detectable clouding). Further, growth of bacteria will also cause changes in transparency and turbidity, thereby enabling straight-forward detection of contamination.

In another aspect, there is provided a topical formulation for delivery of diclofenac or a pharmaceutically acceptable salt thereof, comprising an oily phase and a gelled aqueous continuous phase, the oily phase being in the form of oily domains that are dispersed in the gelled aqueous continuous phase; wherein the oily phase comprises diclofenac or a pharmaceutically acceptable salt thereof, at least one oil, at least two hydrophilic surfactants, at least one co-surfactant, at least two polar solvents, and at least two penetrating promotors; and the gelled aqueous continuous phase comprises an aqueous diluent and at least one gellant.

Diclofenac is a non-steroidal anti-inflammatory drug (NSAID), administered in various dosage forms. In the context of the present disclosure, the term Diclofenac means to encompass 2-(2,6-dichloranilino) phenylacetic acid having the structure shown in formula (I), or any pharmaceutically acceptable salt thereof, including, but not limited to, diclofenac sodium, diclofenac potassium, and diclofenac diethylamine.

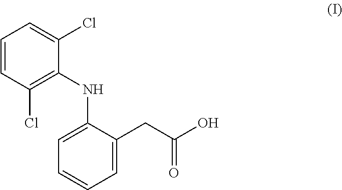 [SMILES: O=C(O)Cc1ccccc1Nc1c(Cl)cccc1Cl]

According to some embodiments, the diclofenac-loaded formulation comprises xanthan gum (such as Xantural 11K, Xantural 180K, Xantural 75 (CP Kelco US) and others (Kelogel or Keltral) as the gellant.

According to other embodiments, the oily phase of the diclofenac-loaded formulation comprises IPM as oil; Tween 60 and HECO 40 as hydrophilic surfactants; IPA, ethanol and PG as polar solvents; a phospholipid as a co-surfactant; DMI and transcutol as penetrating promotors, and optionally one or more fragrance agents, buffers, antioxidants (e.g. BHT) and preservatives.

In another aspect, the present disclosure provides a topical formulation of comprising an oily phase integrated into a gelled aqueous continuous phase, the oily phase being in the form of oily domains dispersed in the continuous gelled aqueous phase, wherein the oily phase comprises an active agent, at least one oil, at least two hydrophilic surfactants, at least one lipophilic co-surfactant, at least two polar solvents, and at least two penetrating promotors, and wherein the gelled aqueous continuous phase comprises an aqueous diluent and at least one gellant; wherein the formulation comprises of at least 2 wt % of diclofenac or a pharmaceutical salt thereof as the active agent.

Other active agents having a structure similar to diclofenac may be loaded into the formulation described herein. Such active agents typically have a main aromatic ring substituted by an amine group. Thus, in some embodiments, the active agent may be selected from compounds having a main aromatic ring substituted by a secondary amine group.

One such active agent is lidocaine, having the structure shown in Formula (II):

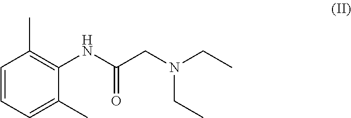 [SMILES: CCN(CC)CC(=O)Nc1c(C)cccc1C]

Another such active agent is clonidine, having the structure shown in Formula (III):

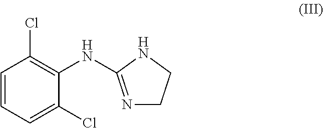 [SMILES: Clc1cccc(Cl)c1NC1=NCCN1]

Yet another such active agent is fentanyl, having the structure shown in Formula (IV), or analogues thereof (such as sufentanil, alfentanil, remifentanil, lofentanil, etc.):

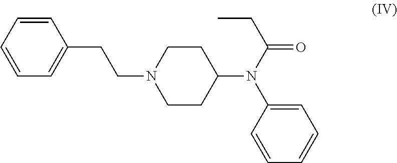 [SMILES: CCC(=O)N(c1ccccc1)C1CCN(CCc2ccccc2)CC1]

A further active agent is trebenifine, having the structure shown in Formula (V), or analogues thereof:

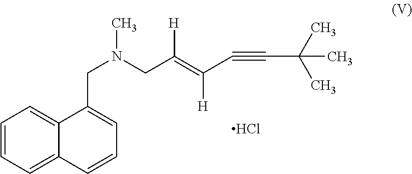 [SMILES: CN(C/C=C/C#CC(C)(C)C)Cc1cccc2ccccc12 ; Cl]

Yet, another active agent is antibiotic cephalexin as can be shown in Formula VI.

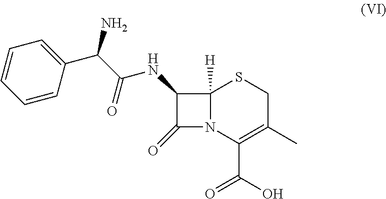 [SMILES: CC1=C(C(=O)O)N2C(=O)[C@@H](NC(=O)[C@H](N)c3ccccc3)[C@H]2SC1]

Yet, another active agent can be sulfamethoxazole shown in formula VII,

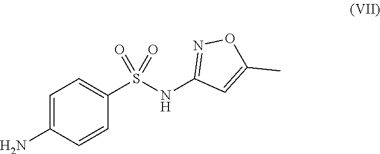 [SMILES: Cc1cc(NS(=O)(=O)c2ccc(N)cc2)no1]

Further active agents may be vancomycin, daptomycin, oritavancin, and tazabactam.

A further active agent, not necessarily consisting aromatic and secondary amino groups, but can be embedded into the interfacial region of the nanodomains is alprostadil (prostaglandin E1), having the structure shown in Formula (VIII), or analogues thereof:

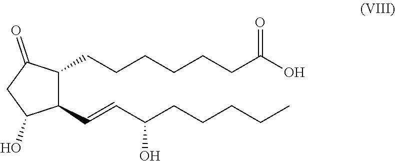 [SMILES: CCCCC[C@H](O)/C=C/[C@H]1[C@H](O)CC(=O)[C@@H]1CCCCCCC(=O)O]

A further active agent is minocycline, having the structure shown in Formula (IX), or analogues thereof:

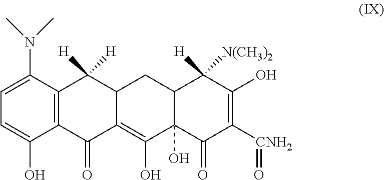

A further active agent is doxycycline, having the structure shown in Formula (X), or analogues thereof:

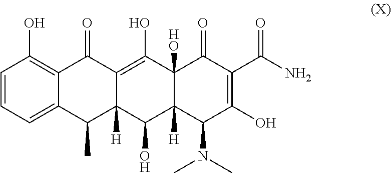 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

Another active agent can be one of a group of anesthetic agent benzocaine or its derivatives as in Formula XI

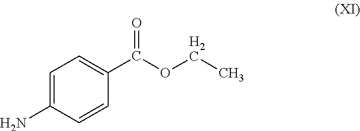

Thus, according to some embodiments, the active agent may be selected from diclofenac, lidocaine, clonidine, fentanyl, trebenifine, alprostadil, sulfamethoxazole, cephalexin, vancomycin, daptomycin, oritavancin, tazabactam, benzocaine, minocycline, doxocycline, or molecules with similar tendency to be incorporated at the domains interface.

This disclosure further provides, in another aspect, a process for preparing a gelled topical formulation as described herein, wherein the process comprises:
(a) providing an active-loaded oily composition comprising at least one active agent, at least one oil, at least two hydrophilic surfactants, at least one co-surfactant, at least two polar solvents, and at least two penetrating promotors, said oily composition being substantially (at times, entirely) devoid of water;
(b) providing an aqueous mixture of an aqueous diluent and at least one gellant; and
(c) mixing the active-loaded oily composition and the aqueous mixture to obtain said gelled topical formulation.

In formulations produced by the processes described herein, the active-loaded oily composition constitutes the oily phase of the formulation, while the aqueous mixture or the gelled aqueous diluent constitutes the gelled aqueous continuous phase.

In some embodiments, the mixing at step (c) is carried out for a period of between about 5 and 60 minutes, and/or at a temperature of between about 25 and 50° C.

In other embodiments, the gellant is present in the aqueous mixture in an amount of between about 0.75 and 3.5 wt %.

It is of note that, in some embodiments, one or more of the process steps may be carried out in a nitrogen atmosphere. In other embodiments, the entire process is carried out under nitrogen atmosphere.

According to some embodiments, the process may comprise adjusting the pH of the formulation, either as a distinct process step or by adding a pH adjusting agent (e.g. buffer) to the aqueous mixture.

According to other embodiments, the process may comprise adding an antioxidant to the formulation, either as a distinct process step or by adding the antioxidant to the active-loaded oily composition.

In another aspect, there is provided a process for preparing a gelled topical formulation as described herein, wherein the process comprises:
(a) providing an active-loaded oily composition comprising at least one active agent, at least one oil, at least two hydrophilic surfactants, at least one co-surfactant, at least two polar solvents, and at least two penetrating promotors, said oily composition being substantially (at times, entirely) devoid of water;
(b) mixing the active-loaded oily composition with an aqueous diluent to obtain a mixture;
(c) adding at least one gellant to the mixture; and
(d) allowing aqueous diluent to gel, thus obtaining said gelled topical formulation.

According to some embodiments, the process may comprise adjusting the pH of the formulation, either as a distinct process step or by adding a pH adjusting agent (e.g. buffer) to the aqueous diluent.

According to other embodiments, the process may comprise adding an antioxidant to the formulation, either as a distinct process step or by adding the antioxidant and/or preservative to the active-loaded oily composition.

According to some embodiments of the processes described herein, step (a) of the process comprises at least two distinct steps: (a1) providing an oily composition that comprises at least one oil, at least two hydrophilic surfactants, at least one co-surfactant, at least two polar solvents and at least one penetrating promotors; and (a2) solubilizing said at least one active agent into the oily composition to obtain said active-loaded oily composition.

Hence, in another aspect, there is provided an oily composition adapted for solubilizing at least one active agent, the oily composition comprises at least one oil, at least two hydrophilic surfactants, at least one co-surfactant, at least two polar solvents, and at least one penetrating promotors, the oily composition being substantially devoid of water.

Namely, in an aspect of this disclosure, there is provided a carrier formulation, substantially devoid of water, being adapted to solubilize at least one active agent, the carrier formulation comprises at least one oil, at least two hydrophilic surfactants, at least one co-surfactant, at least two polar solvents, and at least one penetrating promotors.

In a further aspect, there is provided an active-loaded oily composition comprising at least one active agent, at least one oil, at least two hydrophilic surfactants, at least one co-surfactant, at least two polar solvents, and at least two penetrating promotors, said active-loaded oily composition being substantially (at times, entirely) devoid of water.

The term active-loaded oily composition, interchangeably to be referred to herein as concentrate, denotes a substantially (at times entirely) water-free, oil-based structured lipid/surfactant system, in which surfactant tails solubilize and stabilize the active agent, and the solvent together with the surfactants facilitating full dilution by an aqueous phase (are dilutable) at-will to form the formulation of the invention. In other words, the concentrate is designed for fast and complete dilution in a suitable aqueous medium, which in the process of the invention is viscosified/gelled.

In other words, the nano-domains may be formed in a concentrate form (i.e. a water-free concentrate oily phase), that can be diluted by an aqueous phase at will. Thus, in some embodiments, the concentrates are substantially, at times entirely devoid of water (i.e. water-free).

According to some embodiments, said oil is present in the active-loaded oily composition in an amount of at most 8 wt % (e.g. IPM and the fragrance). The oil may be selected from the oils disclosed herein.

According to other embodiments, said at least two hydrophilic surfactants are present in the active-loaded oily composition in a total amount of at least 22 wt % (e.g. HECO40 and T60). The hydrophilic surfactants may comprise at least a first hydrophilic surfactant in an amount of at least 17.5 wt % (e.g. T60) and a second hydrophilic surfactant in an amount of at least 4.5 wt % (e.g. HECO40); the ratio between the first and second hydrophilic surfactant may, by some embodiments, be between about 5:1 and 2:1 (w/w). The hydrophilic surfactants may each be selected from the surfactants disclosed herein, provided that the first surfactant is different from the second surfactant.

According to some other embodiments, said at least two polar solvents comprise at least a first solvent in an amount of at least 13 wt % (e.g. EtOH and/or IPA) and a second solvent in an amount of at least 22.5 wt % (e.g. PG). The first and second polar solvents may be independently selected from the solvents disclosed herein, provided that the first solvent is different from the second solvent. The ratio between the first and second solvents may, by some embodiments, be between about 1:1.5 and 1:3 (w/w).

In some embodiments, the at least one co-surfactant is present in the active-loaded oily composition in an amount of at least 4.5 wt % (e.g. PC). The at least one co-surfactant may be selected from the co-surfactants disclosed herein.

In some other embodiments, the at least two penetrating promotors are present in the active-loaded oily composition in a total amount of at least 20 wt % (e.g. DMI and transcutol). The penetrating promotors may each be selected from the penetrating promotors disclosed herein.

According to some embodiments, said active agent is present in the active-loaded oily composition in an amount of between 5 and 20 wt %, typically about 10 wt %, 15 wt % or even 20 wt %, and may be selected from diclofenac, lidocaine, clonidine, fentanyl, trebenifine, alprostadil, minocycline, doxocycline, or any pharmaceutically acceptable salt, derivative or analogue thereof.

In some embodiments, the active agent in the concentrate is diclofenac, diclofenac sodium (DCF-Na), diclofenac potassium (DCF-K), diclofenac-ammonium, diclofenac diethylamine (DCF-DEA) and mixtures thereof, or any other pharmaceutically acceptable salt of diclofenac.

This disclosure further provides, in another aspect, a process for preparing a gelled topical formulation for topical delivery of diclofenac or a pharmaceutically acceptable salt thereof, wherein the process comprises:
(a) providing a diclofenac-loaded oily composition comprising diclofenac or a pharmaceutically acceptable salt thereof, at least one oil, at least two hydrophilic surfactants, at least one co-surfactant, at least two polar solvents and at least two penetrating promotors, said oily composition being substantially (at times, entirely) devoid of water;
(b) providing an aqueous mixture of an aqueous diluent and at least one gellant, the gellant being preferably xanthan gum; and
(c) mixing the diclofenac-loaded oily composition and the aqueous mixture to obtain said gelled topical formulation.

In another aspect, there is provided a process for preparing a diclofenac gelled topical formulation as described herein, wherein the process comprises:
(a) providing an diclofenac-loaded oily composition comprising diclofenac or a pharmaceutically acceptable salt thereof, at least one oil, at least two hydrophilic surfactants, at least one co-surfactant, at least two polar solvents, and at least two penetrating promotors, said oily composition being substantially (at times, entirely) devoid of water;
(b) mixing the diclofenac-loaded oily composition with an aqueous diluent to obtain a mixture;
(c) adding at least one gellant to the mixture; and
(d) allowing aqueous diluent to gel, thus obtaining said gelled topical formulation.

In some embodiments of the processes described herein, the formulation may comprise at least one additional component selected from at least one buffer and at least one pH adjusting agent, antioxidant and preservative.

According to another aspect, the invention provides a method of topically delivering an active agent to a subject in need thereof, comprising topically administering to the subject an effective amount of the formulation described herein.

In another aspect, there is provided a method of topically delivering diclofenac or a pharmaceutically acceptable salt thereof to a subject in need thereof, comprising topically administering to the subject an effective amount of the formulation described herein.

As known, the “effective amount” for purposes herein may be determined by such considerations as known in the art. The effective amount is typically determined in appropriately designed clinical trials (dose range studies) and the person versed in the art will know how to properly conduct such trials in order to determine the effective amount. As generally known, the effective amount depends on a variety of factors including the distribution profile within the body, a variety of pharmacological parameters such as half-life in the body, on undesired side effects, if any, on factors such as age and gender, and others.

The term “subject” refers to a mammal, human or non-human.

In another aspect, there is provided a formulation as described herein for use in treating a disease or condition in a patient or individual in need thereof.

The formulations according to the invention may be used to induce at least one therapeutic effect, i.e. inducing, enhancing, arresting or diminishing at least one effect, by way of treatment or prevention of unwanted conditions or diseases in a subject. The term treatment or any lingual variation thereof, as used herein, refers to the administering of a therapeutic amount of the formulation disclosed herein which is effective to ameliorate undesired symptoms associated with a disease or condition, to prevent the manifestation of such symptoms before they occur, to slow down the progression of the disease or condition, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

In some embodiments, said disease or condition is selected from an inflammatory disease, mild to moderate pain, swelling, musculoskeletal disorders, or sign and symptoms of osteoarthritis, joint stiffness or rheumatoid arthritis, as well as inflammatory skin conditions.

In another aspect, the invention provides a kit comprising the formulation as described herein in a dosing form and instructions for use.

The term “dosing form” refers to a compartment or a container or a discrete section of a vessel, for holding or containing the formulation. Within the context of the present invention, the term also refers to separate containers or vessels, housed within a single housing.

Each one of the containers may be of single or multiple-dose contents. The containers may be in any form known in the art, such as vial, ampoules, collapsible bags, tube, spray, roll-on, container associated with a pumping and/or dispensing means, swabs, pads absorbed with the formulation, etc., enabling application of the formulation to a desired skin area.

In some embodiments, the kit may comprise at least one measuring tool, for measuring the weight, volume or concentration of each component.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

As used herein, the term “about” is meant to encompass deviation of 10% from the specifically mentioned value of a parameter, such as temperature, pressure, concentration, etc.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows LUMiFuge™ test results of commercial emulsion for 17 hr at 3000 rpm, showing that a commercial emulsion does not maintain transparency nor stability for long periods of time.

FIG. 2 shows the effect of changing the phospholipid component on the transparency of 2 wt % DCF-Na loaded gelled formulation.

FIG. 3 shows the effect of increasing the gellant content on the transparency of 2 wt % DCF-Na loaded gelled formulation.

FIG. 4 shows the effect of changing the perfuming agent on the transparency of 2 wt % DCF-Na loaded gelled formulation.

FIGS. 5A-5B show the effect of dilution on unloaded and 2 wt % DCF-Na loaded formulation as measured by electrical conductivity tests, respectively.

FIGS. 6A-6B show non-gelled formulation un-loaded and loaded with DCF-Na, respectively at different water dilutions, respectively.

FIG. 7 shows non-gelled formulation loaded with lidocaine at different water dilutions.

FIGS. 8A-8D are cryo-TEM micrographs of non-gelled formulation A of Table 2 (×650K magnification): 80 wt % water, unloaded with DCF-Na (FIG. 8A); 80 wt % water, 2 wt % DCF-Na (FIG. 8B); 90 wt % water, unloaded with DCF-Na (FIG. 8C); and 90 wt % water, 2 wt % DCF-Na (FIG. 8D).

FIG. 9 is a cryo-TEM micrograph of a gelled 2 wt % DCF-Na loaded formulation.

FIGS. 10A-10D are SAXS measurements of Formulation A in Table 2 at various storage temperatures and duration: freshly made (FIG. 10A); stored at 5° C. for 2 weeks (FIG. 10B); stored at 5° C. for 6 months (FIG. 10C); and stored at 25° C. for 6 months (FIG. 10D).

FIG. 11A shows the diffusion coefficients (Dx, m²/sec) of the main components for un-loaded and 2 wt % DCF-Na loaded formulation, 80 wt % water dilution, in non-gelled and gelled systems (0.75 wt % gellant). FIG. 11B shows the diffusion coefficients of the main components of a 2 wt % DCF-Na at various water dilutions.

FIG. 12 shows a comparison of visual appearance of a 2 wt % DCF-Na gelled formulation, 80 wt % water (named NDS 506(A)) (right) and Voltaren Emulgel® (left).

FIGS. 13A-13B show polarized light microscopic images of Voltaren Emulgel® (FIG. 13A) and NDS 506(A) (FIG. 13B), magnification ×10.

FIGS. 14A-14B show oily domains size distribution of gelled DCF-Na formulation, as measured by DLS (Dynamic Light Scattering) analysis; water concentration being 80 wt % (FIG. 14A) and 90 wt % (FIG. 14B), as measured without the addition of a gelling agent.

FIGS. 15A-15B show rheological behavior tests of stress r (Pa) as a function of shear rate 7 (1/s) of Voltaren Emulgel® (FIG. 15A) and NDS 506(A) (FIG. 15B).

FIG. 16 shows viscosity measurements at constant sheer rate at 50 Hz, against time (sec) for gelled aqueous phase (without an oily phase) and for gelled DCF-Na loaded formulations for various xanthan contents (0.75%, 0.85% and 1.0%,).

FIG. 17A shows the dynamic complex viscosity of the flow p of the gelled aqueous phase (without an oily phase) against the shear rate (1/s) and gelled 2 wt % DCF-Na loaded formulation; FIG. 17B shows the viscosity of aqueous phase (without an oily phase), an un-loaded gelled formulation and 2 wt % DCF-Na loaded gelled formulation over time at a constant shear rate.

FIG. 18A shows the storage and loss moduli (G′, G″) for gelled aqueous phase (without an oily phase) and gelled 2 wt % DCF-Na loaded formulation; and FIG. 18B shows the storage and loss moduli (G′, G″) for un-loaded and 2 wt % DCF-Na loaded gelled formulations.

FIG. 19 shows complex viscosity measurements for NDS 506(A) formulation with various xanthan concentrations (ranging from 0.75 wt % to 2.85 wt %) compared to Voltaren Emulgel®.

FIGS. 20A-20D show spreadability test results for Voltaren Emulgel® Forte (FIGS. 20A-20B) and formulation NDS 506(A) (FIGS. 20C-20D).

FIG. 21 show ex vivo penetration and permeation after 24 hours (% Na-DCF form applied dose) Franz cell diffusion tests results carried out on pig skin samples, comparing between NDS 506(A) and Voltaren Emulgel® Forte.

FIG. 22 shows penetration profiles of DCF-Na concentration (g/cm²) for NDS 506(A), viscosified with 0.75 wt % or 2.85 wt % of xanthan gum.

FIGS. 23A-23B show LUMiFuge™ test results for NDS 506(A) (FIG. 23A) and typical commercial emulsion (FIG. 23B).

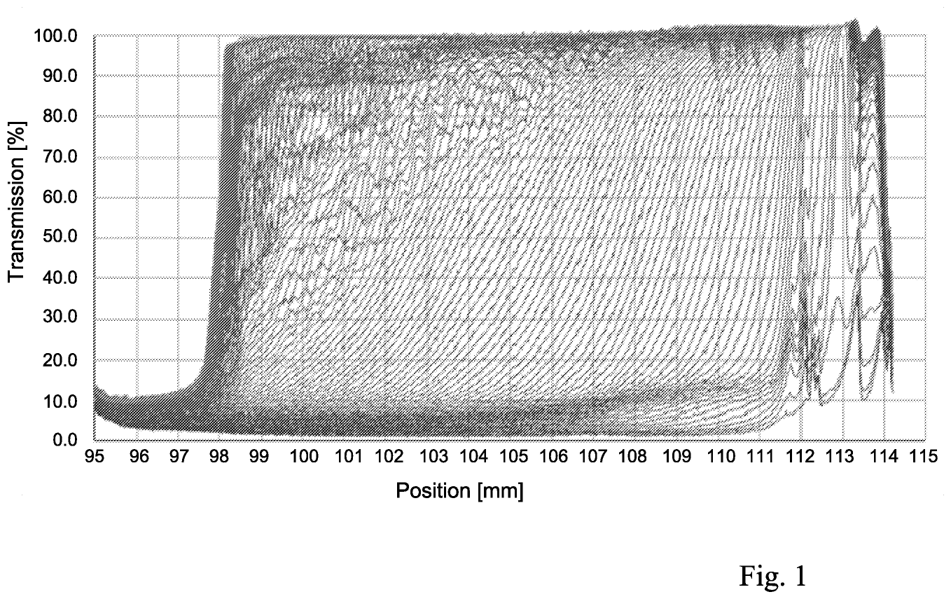

### DETAILED DESCRIPTION OF EMBODIMENTS

### Preparation of an Active-Loaded Gelled Formulation

### Step 1: Preparation of Concentrate or Oily Phase

An excipient mixture was prepared by mixing phosphatidylcholine phospholipid (PC) (preheated to 45° C. until full melting), hydrogenated castor oil (40EO), Tween 60, propylene glycol (PG), isopropyl myristate (IPM), transcutol, dimethyl isosorbide (DMI), fragrance, ethanol (EtOH), and isopropyl alcohol (IPA). The mixture was thoroughly mixed at 300-600 RPM at 25° C. The mixture resulted in a clear, transparent yellowish liquid.

The active compound was added in powdered form to the mixture and mixed for 10-30 minutes to obtain full entrapment of the active agent.

### Step 2: Preparation of Active-Loaded Gelled Formulation

The active-loaded oily composition may be diluted with any desired amount of water in order to obtain a desired concentration of the active. Typically, the concentrate is diluted by adding between 70 to 90 wt % of water.

In order to obtain the gelled formulation, xanthan gum was dissolved into purified water that was buffered to pH of 7.2-7.4 by gentle mixing to obtain homogeneity without lumps of xanthan gel.

The xanthan gel was added to the loaded oily composition under mixing conditions at room temperature, with gentle mixing until uniform, almost clear gel is formed. The formulation is placed under vacuum or centrifugation to remove any bubbles that may have been entrapped in the final product having spontaneously formed oily-phase domains having a size of <20 nm within the gelled aqueous phase.

In another sequence of preparation, Heco40 and Tween 60 are heated to 45° C. and allowed to fully melt. The temperature is lowered and PG, IPA, ethanol, IPM, transcutol, DMI, fragrance, and optionally antioxidant are added and mixed to obtain a clear solution. The PC is then added to the oily mixture, and optionally heated to 45° C. to allow full integration of the PC into the oily phase. The system is cooled to room temperature and then powdered Na-DCF is added stepwise into the oily phase to form a concentrate.

The gelled aqueous phase is prepared by dissolving the xanthan gum in purified buffered aqueous solution or purified water in which pH was adjusted to the desired pH. The concentrate is then added to the aqueous phase at room temperature, under mixing until uniform homogeneous almost clear gel is formed. The formulation is placed under vacuum or centrifugation to remove any bubbles that may have been entrapped in the final product.

The resulting system in a diluted gelled formulation with the spontaneously formed oily-phase domains having a size of <20 nm dispersed within the gelled aqueous phase.

The composition of the active-loaded gelled formulation is provided in Table 1.

TABLE 1
Diluted gelled active-loaded formulation

| Component | Function | Amount (wt %) |
|---|---|---|
| Lecithin (PC) | Phospholipid, | 0.5 to 1.5 |
| | lipophilic co- | |
| | surfactant | |
| Tween 60 | First hydrophilic | 3.0 to 5.0 |
| | surfactant | |
| Hydrogenated castor oil | Second hydrophilic | 0.6 to 1.5 |
| (40EO) | surfactant | |
| Propylene glycol (PG) | Co-surfactant/solvent | 2.0 to 6.0 |
| Isopropyl myristate (IPM) | Oil | 1.0 to 4.0 |
| Transcutol | Solvents and/or | 1.5 to 3.5 |
| Dimethyl isosorbide (DMI) | penetrating promoters | 0.9 to 3.0 |
| Peppermint oil | Fragrance/Oil/ | 0.2 to 0.6 |
| | Penetrating promoter | |
| Ethanol (EtOH) | Polar solvent | 1.5 to 2.5 |
| Isopropyl alcohol (IPA) | | 1.5 to 2.5 |
| Xanthan gum | Viscosifier/gellant | 0.75 to 3.0 |
| Water | — | 60-90 |
| Active agent | API | 1.0-5.0 |

### Variance in Formulation

Table 2 shows some additional exemplary formulations according to this disclosure, including variations of the formulations that include, inter alia, antioxidants (for example BHT).

TABLE 2
Exemplary formulations (all amounts are given in wt % out of the formulation)

| Component | A | B | C | D | E | F | G |
|---|---|---|---|---|---|---|---|
| Lecithin (PC) | 0.90 | 0.90 | 0.90 | 0.90 | — | — | 0.90 |
| Ethoxylated castor oil (HECO-40) | 0.90 | 0.90 | 0.90 | 0.90 | 0.90 | 0.90 | 0.90 |
| Propylene glycol (PG) | 3.50 | 3.50 | 3.50 | 3.50 | 3.50 | 3.50 | 3.50 |
| Tween 60 (Tw60) | 4.50 | 4.50 | 4.50 | 4.50 | 5.40 | 4.50 | 4.48 |
| Iso propyl mirystate (IPM) | 1.00 | 1.00 | 1.00 | 1.00 | 1.00 | 1.00 | 1.00 |
| Dimethyl isosorbide (DMI) | 1.60 | 1.60 | 1.60 | 1.60 | 1.60 | 1.60 | 1.60 |
| Diethylene glycol | 2.40 | 2.40 | 2.40 | 2.40 | 2.40 | 2.40 | 2.40 |
| monoethyl ether (TC) | | | | | | | |
| Perfume | 0.60 | — | 0.60 | — | 0.60 | 0.60 | 0.60 |
| Ethanol (EtOH) | 1.30 | 1.30 | 1.30 | 1.30 | 1.30 | 1.30 | 1.30 |
| Isopropyl alcohol (IPA) | 1.30 | 1.90 | 1.30 | 1.30 | 1.30 | 2.20 | 1.30 |
| Diclofenac sodium (API) | 2.00 | 2.00 | 2.00 | 2.00 | 2.00 | 2.00 | 2.00 |
| Water | 79.25 | 79.25 | 79.25 | 79.25 | 79.25 | 79.25 | 79.25 |
| Xanthan gum | 0.75 | 0.75 | 0.75 | 0.75 | 0.75 | 0.75 | 0.75 |
| Butylated hydroxytoluene (BHT) | — | — | — | — | — | — | 0.02 |
| Component | H | I | J | K | L | M | N |
| Lecithin (PC) | 0.90 | 0.90 | 0.90 | 0.90 | 0.90 | 0.90 | 0.90 |
| Ethoxylated castor oil (HECO-40) | 0.90 | 0.90 | 0.90 | 0.90 | 0.90 | 0.90 | 0.90 |
| Propylene glycol (PG) | 3.50 | 3.50 | 3.50 | 3.50 | 3.50 | 3.50 | 3.50 |
| Tween 60 (Tw60) | 11.63 | 4.50 | 4.50 | 4.50 | 4.90 | 4.50 | 4.50 |
| Iso propyl mirystate (IPM) | 1.00 | 1.00 | 1.00 | 1.00 | 1.00 | 1.00 | 1.00 |
| Dimethyl isosorbide (DMI) | 1.60 | 1.60 | 1.60 | 1.60 | 1.60 | 1.60 | 1.60 |
| Diethylene glycol | 2.40 | 2.40 | 2.40 | 2.40 | 2.40 | 2.40 | 2.40 |
| monoethyl ether (TC) | | | | | | | |
| Perfume | 0.60 | 0.60 | 0.60 | 0.60 | 0.20 | 0.60 | 0.60 |
| Ethanol (EtOH) | 1.30 | 1.30 | 1.30 | — | 1.30 | 1.30 | 1.30 |
| Isopropyl alcohol (IPA) | 8.43 | 15.55 | 15.55 | 2.60 | 1.30 | 1.30 | 1.30 |
| Diclofenac sodium (API) | 2.00 | 2.00 | 2.00 | 2.00 | 2.00 | 2.00 | 2.00 |
| Water | 65.00 | 65.00 | 65.00 | 79.25 | 79.25 | 79.00 | 78.50 |
| Xanthan gum | 0.75 | 0.75 | 0.75 | 0.75 | 0.75 | 1.00 | 1.50 |
| Butylated hydroxytoluene (BHT) | — | — | — | — | — | — | — |

| Component | O | P | Q | R | S |
|---|---|---|---|---|---|
| Lecithin (PC) | 0.90 | 0.90 | 0.90 | 1.35 | 0.80 |
| Ethoxylated castor oil (HECO-40) | 0.90 | 0.90 | 0.90 | 1.35 | 0.80 |
| Propylene glycol (PG) | 3.50 | 3.50 | 3.50 | 5.25 | 3.40 |
| Tween 60 (Tw60) | 4.50 | 4.50 | 3.50 | 6.25 | 4.40 |
| Iso propyl mirystate (IPM) | 1.00 | 1.00 | 1.00 | 1.50 | 0.90 |
| Dimethyl isosorbide (DMI) | 1.60 | 1.60 | 1.60 | 2.40 | 1.50 |
| Diethylene glycol | 2.40 | 2.40 | 2.40 | 3.60 | 2.30 |
| monoethyl ether (TC) | | | | | |
| Perfume | — | — | — | — | — |
| Peppermint oil | 0.60 | 0.60 | 0.60 | 0.90 | 0.50 |
| Ethanol (EtOH) | 1.30 | 1.30 | 1.30 | 1.95 | 1.20 |
| Isopropyl alcohol (IPA) | 1.30 | 1.30 | 1.30 | 1.95 | 1.20 |
| Diclofenac sodium (API) | 2.00 | 2.00 | 3.00 | 3.00 | 3.00 |
| Water | 78.75 | 77.15 | 77.15 | 69.75 | 79.25 |
| Xanthan gum | 2.00 | 2.85 | 2.85 | 0.75 | 0.75 |
| Butylated hydroxytoluene | — | — | — | — | — |
| (BHT) | | | | | |

All the formulations in Table 2 were obtained by mixing the ingredients according to the processes described herein. The resulting formulations were clear and transparent, without any evidence of phase separation or droplets coalescence.

Incorporation of various perfuming agents, antioxidants and/or pH adjusting agents (buffers) did not change the nanostructure of the formulation.

### Variance in Type of Phospholipid

The influence of changing the phospholipid components on the formulations of the invention was tested for Formulation A of Table 2. Various sources of phosphatidyl choline (PC) from various lecithin derivatives and PC levels ranging from 70% to 94% were tested:
Lipoid-S75 (70% PC), Lipoid-S100 (94% PC), Phospholipon 90G (94% PC), Epicorn 200 (94% PC) are soy-based;
Lipoid-P100 GMO-free, 90% PC from soybean;
Lipoid-H100 GMO-free, 90% PC from sunflower seed; and
Lipoid-R100 GMO-free, 90% PC from rapeseed.

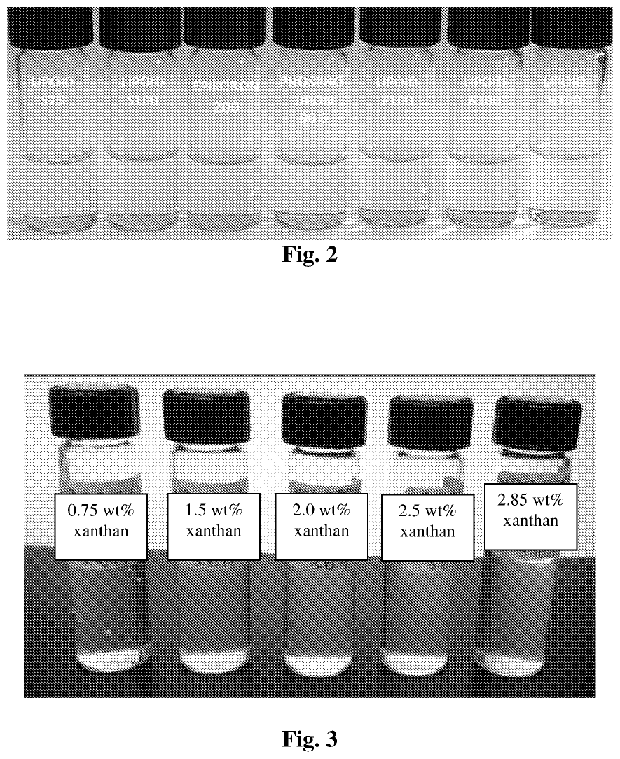

As seen in FIG. 2, all of the phospholipid tested resulted in clear and transparent formulations, without evidence of phase separation or droplets coalescence.

### Variance in Type and Amount of Gellant

The influence of changing the type of gellant on the formulations of the invention was tested for Formulation A of Table 2. Various types of xanthans were tested, at 2 concentrations: 1 wt % and 0.75 wt % out of the formulation. Table 3 presents characterization of the gelled formulations tested with three different xanthans (Xantural® 75, 180 and 11K, all provided by PC Kelco).

TABLE 3
Characterization of Formulation A gelled with different gellants

| | | | | | | Viscosity | | |
|---|---|---|---|---|---|---|---|---|
| | | | | | | (mPas) | | |
| | | | Micros- | Turbidity | | 0.75 | 1 | |
| # | Xanthan type | Appearance | copy | (NTU) | pH | wt % | wt % | LUMiFuge* |
| 1 | Xantural 75 | Transparent | Clear | 45 | 7.25 | 109.6 | 165 | Good |
| 2 | Xantural 180 | Transparent | Clear | 45 | 7.16 | 119.2 | 172.1 | Good |
| 3 | Xantural 11K | Transparent | Clear | 25 | 7.12 | 116.1 | 169.1 | Good |

*see explanation about the LUMiFuge ™ test further below.

As can be seen, the formulations maintain their properties when varying the type of xanthan used as a gellant.

The influence of the amount of gellant was also assessed. Based on Formulation A in Table 2, the amount of xanthan (Xantural 11K) was varied between 0.75 wt % and 2.85 wt %. The pH, turbidity and long-term stability were measured for these formulations as shown in Table 4 (and FIG. 3).

TABLE 4
Characterization of formulation A with varying amount of xanthan

| | Xanthan (wt %) | | | | |
|---|---|---|---|---|---|
| | 0.75 | 1.5 | 2.0 | 2.5 | 2.85 |
| pH | 6.85 | 6.85 | 6.78 | 6.73 | 6.77 |
| Turbidity | 20 | 24 | 33 | 37 | 75 |
| (NTU) | | | | | |
| LUMiFuge | Good | Good | Good | Good | Good |

Although increasing the amount of xanthan, all formulations remained transparent, without any significant change in pH or turbidity. No changes in transparency of the formulations was detected in LUMiFuge™ tests, indicating that increasing the amount of xanthan does not damage the long-term stability of the formulation.

### Variance in Perfuming Agent

As perfuming agents are typically oil-based and oil-soluble, the effect of the presence or absence of perfume on the nano-structure and the stability of the formulation was testes, as well as the effect of variance in the type of perfume. Table 5 details the compositions of the tested formulations, all based on Formulation A in Table 2, from which 0.6 wt % is a varying perfume.

TABLE 5
Formulation A with various perfuming agents

| | Composition | | DLS** | | | |
|---|---|---|---|---|---|---|
| Formu- | | Completing | Size | Volume | | Turbidity |
| lation | Perfume | component* | (nm) | (%) | PDI | (NTU) *** |
| A | 0.6 wt % Perfume 1 | — | 6.395 | 100 | 0.197 | 30 |
| AB | — | 0.6 wt % PG | 7.649 | 100 | 0.567 | 40 |
| AC | — | 0.6 wt % water | 6.930 | 100 | 0.554 | 32 |
| AD | 0.2 wt % Perfume 1 | 0.4 wt % PG | 6.993 | 100 | 0.295 | 50 |
| AE | 0.1 wt % Perfume 2 | 0.5 wt % IPA | 6.545 | 100 | 0.312 | 60 |

*Formulation A contained 0.6 wt % of perfume 1; the completing component refers to the component added to the formulation when reducing or eliminating the perfume.
**Tested by DLS Zeta sizer by Malvern, Model ZEN1600; due to the nature of the test, DLS measurements were carrying out on non-gelled formulations.
*** Tested by Turbidity HANNA Instrument, model HI183414 (230VAC/50 Hz/10VA- Fuse 400 mA).

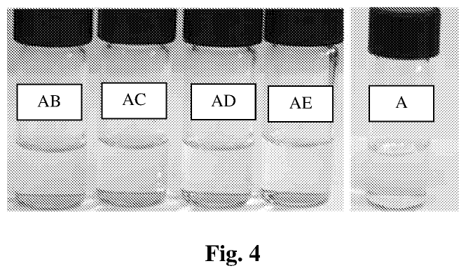

As evident by Table 5 and FIG. 4, replacing the perfume agent and/or eliminating the perfume agent from the formulation does not affect its transparency. Optical microscopy and DLS measurements revealed that no change in nanostructure was visible: the samples remain clear (transparent), without any visible change in turbidity. In all samples, the nanodomain size measured with non-gelled system was maintained below 10 nm (monodispersed), without any evidence of phase separation or coalescence of the nanodomains, indicating good compatibility of the nanodomains and different fragrances. This suggest that the perfumes, which are oil-soluble, are solubilized in the core of the droplets and well integrated into the interphase. Stability and transparency was gained with the gelled systems as well.

This is also supported by the SD-NMR measurements carried out for the examples that are shown in Table 6. No significant changes in diffusion coefficients were measured, meaning that the active agent (DCF-Na) is maintained at the interphase although replacing or eliminating the oil-soluble perfume agent.

TABLE 6
SD-NMR* results for formulations with different perfumes

| | Diffusion coefficient × 10⁻⁹ (m²s⁻¹) | | | | |
|---|---|---|---|---|---|
| Component | A | AB | AC | AD | AE |
| Surfactants | 0.01 | 0.01 | 0.01 | 0.01 | 0.01 |
| Co-surfactant | 0.50 | 0.56 | 0.59 | 0.55 | 0.59 |
| Water | 1.50 | 1.55 | 1.52 | 1.48 | 1.48 |
| DCF-Na | 0.1 | 0.1 | 0.1 | 0.1 | 0.1 |

*see detailed explanation about the SD-NMR measurement technique further below.

As an indicator to the long term stability of the formulations, LUMiFuge™ measurements were carried out for 17 hr at 3000 rpm, and full transparency of the samples was maintained over the entire duration of the test. These conditions are comparable to 3 years of storage, indicating that changing the perfume agent or eliminating it from the formulation is will not influence the long term stability of the formulations.

### Diclofenac Sodium (DCF-Na) Loaded Zelled Formulation

### Effect of Dilution on the Oily Phase Structure

2 wt % DCF-Na loaded oily composition which is substantially devoid of water (i.e. a concentrate) was prepared according to the process described above. The un-diluted oily composition was constituted by self-assembled oil-solvated clusters or short domains of surfactants, which differ from the classical reverse micelles. These concentrates are dilutable by any suitable diluent, for example by purified water, to form a diluted delivery system.

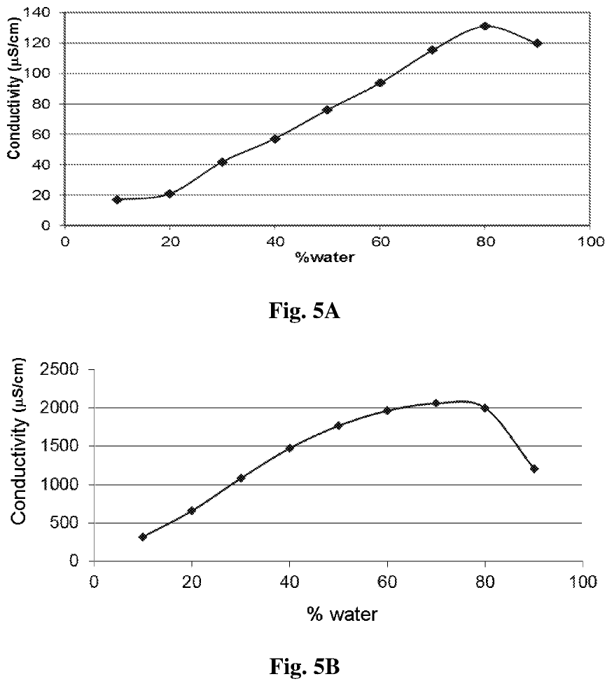

The effect of water dilution on the oily domains structure was investigated by using electrical conductivity tests. Electrical conductivity measurements were performed at 25±2° C. using a conductivity meter, type CDM 730 (Mettler Toledo GmbH, Greifensee, Switzerland). Measurements were made on empty and DCF-Na loaded samples upon dilution with water up to 90 wt %. No electrolytes were added to the samples. The conductivity allowed the identification of the continuous phase and the inner phase. The results are shown in FIGS. 5A-5B.

The oily domains undergo phase transitions upon increasing the amount of diluent (e.g. water). When in the concentrated form, the oily composition is in the form of oil solved clusters (short surfactant domains), such that DCF-Na resides within the oil domains. When mixed with increasing amounts of water, hydrated domains are formed; upon further dilution with water, structure progressively and continuously transforms into oily domains dispersed in water, such that the DCF-Na molecules are located and entrapped by the tails of the surfactants at the interface of the oily domains with the water phase. It is of note that the absolute values of the conductivity of the empty system are significantly lower than those of the loaded system due to the ionic nature of DCF-Na.

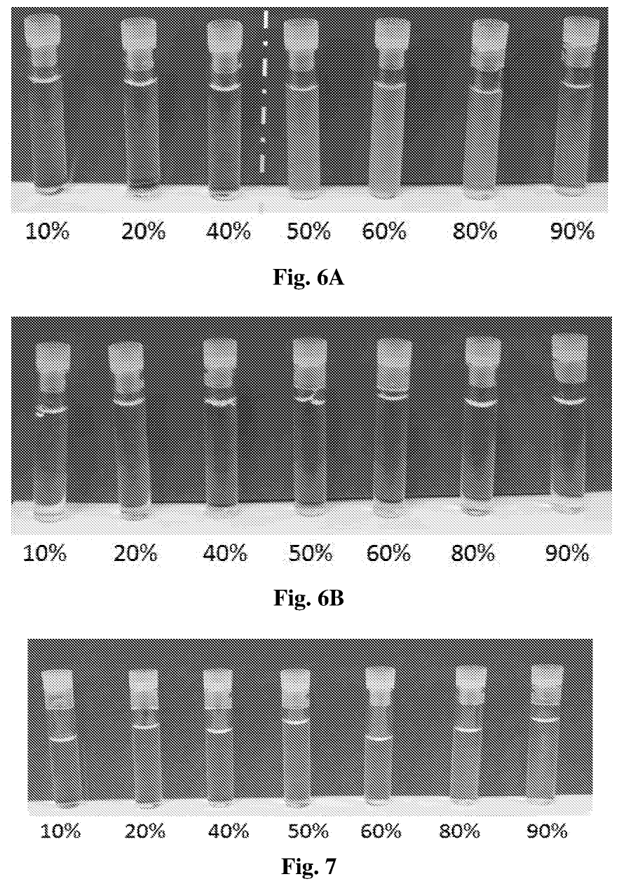

It was noted that the oily carrier, i.e. the oily composition without DCF-Na, could not be fully diluted. Only upon addition of the DCF-Na, stable oily domains were obtained, as seen in FIGS. 6A and 6B. In FIG. 6A, un-loaded oily phase was diluted to various water concentrations; as can be seen, above 50 wt % water, the system phase separates. When the oily phase was loaded with 2 wt % of DCF-Na, the system was fully dilutable up to 90 wt %, resulting in a clear and transparent formulation, as seen in FIG. 6B.

As seen in FIG. 7, similar results were obtained when the oily phase was loaded with lidocaine, a structure builder similar in function to that of Na-DCF.

This attests to the function of the active agent in stabilizing the oily domains interface; the active agent functions as a structurant, contributing and facilitating the final structure of the oily domains. This behavior differs from classic carrier systems, in which the active agent is merely loaded into the formulation, without taking part of the actual structure of the system. Thus, throughout the phase transformations occurring upon dilution, DCF-Na stabilized the structure of the delivery system and is entrapped within the interface, (as will be further explained below in connection with SD-NMR analysis).

Additional formulations with various dilution levels are shown in Tables 7-1 and 7-2.

TABLE 7-1
Formulations with various water-dilution levels (between 1 and 4 wt % DCF)

| | Dilution factor | | | | | | |
|---|---|---|---|---|---|---|---|
| | 1.00 | 10.00 | 5.00 | 4.00 | 3.33 | 2.86 | 2.50 |
| Lecithin (PC) | 4.5 | 0.45 | 0.9 | 1.13 | 1.35 | 1.58 | 1.8 |
| Ethoxylated castor oil (HECO-40) | 4.5 | 0.45 | 0.9 | 1.13 | 1.35 | 1.58 | 1.8 |
| Propylene glycol (PG) | 22.5 | 2.25 | 4.5 | 5.63 | 6.75 | 7.88 | 9.0 |
| Tween 60 (Tw60) | 17.5 | 1.75 | 3.5 | 4.38 | 5.25 | 6.13 | 7.0 |
| Iso propyl mirystate (IPM) | 5 | 0.5 | 1.0 | 1.25 | 1.5 | 1.75 | 2 |
| Dimethyl isosorbide (DMI) | 1.60 | 0.8 | 1.6 | 2 | 2.4 | 2.8 | 3.2 |
| Diethylene glycol | 12 | 1.2 | 2.4 | 3. | 3.6 | 4.2 | 4.8 |
| monoethyl ether (TC) | | | | | | | |
| Perfume | 0.5 | 0.05 | 0.1 | 0.13 | 0.15 | 0.18 | 0.2 |
| Ethanol (EtOH) | 6.5 | 0.65 | 1.3 | 1.63 | 1.95 | 2.28 | 2.6 |
| Isopropyl alcohol (IPA) | 9 | 0.9 | 1.8 | 2.25 | 2.7 | 3.15 | 3.6 |
| Diclofenac sodium (API) | 10 | 1 | 2 | 2.5 | 3 | 3.5 | 4 |
| Water | 0 | 87.15 | 77.15 | 72.15 | 67.15 | 62.15 | 57.15 |
| Xanthan gum | 0 | 2.85 | 2.85 | 2.85 | 2.85 | 2.85 | 2.85 |

TABLE 7-2
Formulations with various water-dilution levels (for 2 wt % DCF)

| | | | | | | | | | |
|---|---|---|---|---|---|---|---|---|---|
| Lecithin | 4.5 | 0.45 | 0.9 | 1.13 | 1.35 | 1.58 | 1.8 | 1.8 | 1.8 |
| HECO-40 | 4.5 | 0.45 | 0.9 | 1.13 | 1.35 | 2.58 | 2.8 | 1.8 | 3.8 |
| PG | 22.5 | 2.5 | 5 | 6.25 | 7.5 | 8.25 | 10 | 11 | 5 |
| Tw60 | 17.5 | 1.75 | 3.5 | 4.38 | 3.4 | 6.13 | 7 | 7 | 8 |
| IPM | 5 | 0.5 | 1.0 | 1.25 | 1.5 | 1.75 | 2 | 2 | 2 |
| DMI | 1.60 | 0.8 | 1.6 | 2 | 2.4 | 2.8 | 3.2 | 3.2 | 3.2 |
| TC | 12 | 1.2 | 2.4 | 3 | 3.6 | 4.2 | 6.8 | 4.8 | 4.8 |
| Perfume | 0.5 | 0.05 | 0.1 | 0.13 | 0.15 | 0.18 | 0.2 | 0.2 | 0.2 |
| Ethanol | 6.5 | 0.65 | 1.3 | 1.63 | 1.95 | 2.28 | 2.75 | 2.6 | 2.6 |
| IPA | 9 | 0.65 | 1.3 | 2.13 | 1.95 | 2.43 | 2.6 | 3.6 | 3.6 |
| DCF-Na | 10 | 2 | 2 | 2 | 2 | 2 | 2 | 2 | 2 |
| Water | 0 | 86.15 | 77.15 | 72.15 | 70 | 63 | 56 | 57.15 | 60.15 |
| Xanthan | 0 | 2.85 | 2.85 | 2.85 | 2.85 | 2.85 | 2.85 | 2.85 | 2.85 |

### Structure of Nanodomains

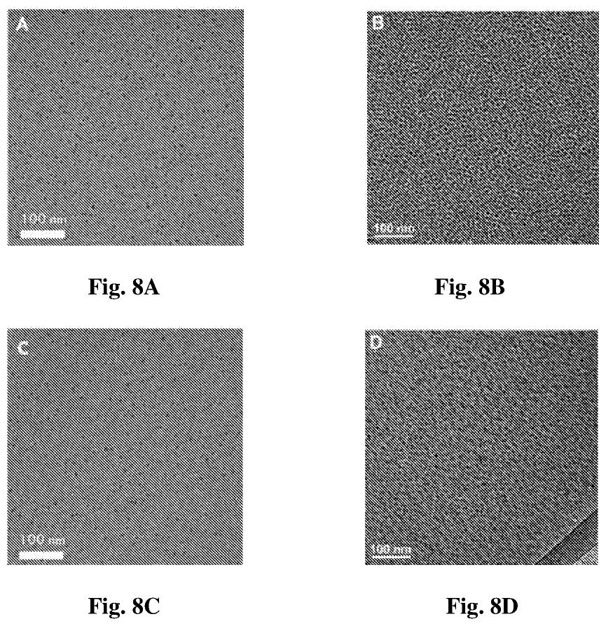

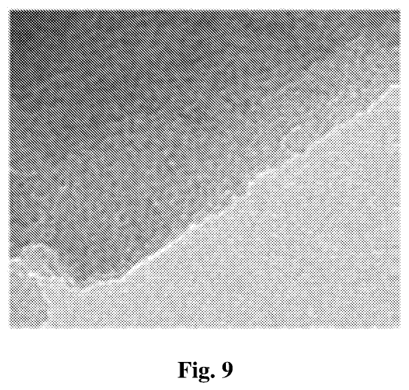

Photomicrographs of diluted formulations (×650K magnification, FIGS. 8A-8D) indicate that the domains are almost mono dispersed in size. The domains are not necessarily spherical and consist of an oily core and an interface comprising surfactants and co-surfactants. The domains are dispersed in aqueous continuous phase. While the empty droplets (FIGS. 8A and 8C) are more spherical, the loaded systems (FIGS. 8B and 8D) have droplets with substantially elongated shape with an aspect ratio of 1.1 to 1.5. Upon further dilution (i. e. increasing the dilution from 80 wt % water to 90 wt % water) the droplets become less packed and smaller in number per volume.

As seen in FIG. 9, although the formulation is gelled, the nanodomains remain structured, meaning that the solubilization capacity, stability and release profiles are not affected by the formation of a viscoelastic network in the aqueous phase. In other words, the gelling process of the aqueous phase does not affect the structure and stability of the nanodomains.

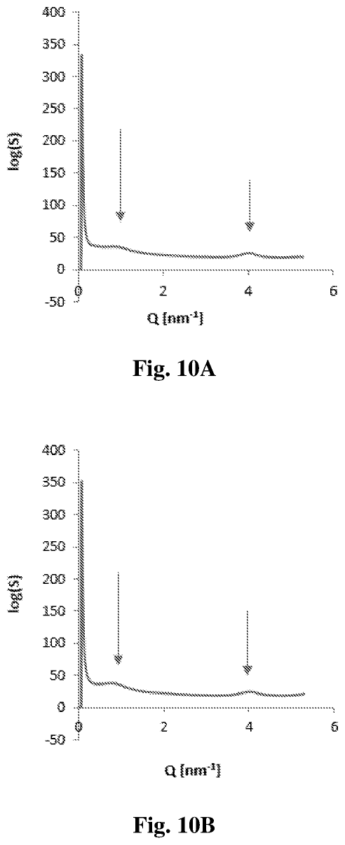

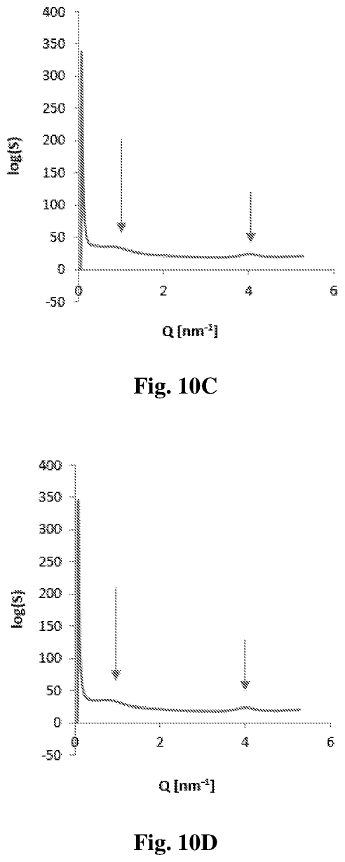

Small-Angle X-ray Scattering (SAXS) measurements suggest that the domains are well structured with almost constant size and distance between droplets (lattice parameters), which do not change over time or temperature (FIGS. 10A-10D). All samples measured have shown similar domains sizes, ranging from 7.1 nm to 8.6 nm with a distance of ca. 1.6 nm between droplets.

When comparing the unloaded system with the DCF-loaded system, it seems that the presence of DCF-Na allows to obtain smaller oily domains; namely, when DCF-Na was loaded into the system, smaller and more uniform domains were spontaneously obtained (16 nm vs. 6-10 nm for un-loaded and loaded oily phases, respectively). This also attests to the function of the DCF-Na as a structurant (functioning as a cosmotropic agent), as shown in Table 8.

TABLE 8
Oily domains average size values

Domain size (nm)

| Water content (wt %) | Unloaded | DCF-Na loaded |
|---|---|---|
| 80 | 16.3 (±1.3) | 5.8 (±0.6) |
| 90 | 15.9 (±0.4) | 6.7 (±0.3) |

Upon adding the gellant to the formulation, the structure is further modified and larger oily domains are formed. These domains are not spherical and their average size increased (via estimated measurements) to about 10-15 nm, as detailed in Table 9.

TABLE 9
Oily domains average size values in
DCF-Na loaded gelled formulations

Domain size (nm)

| Water content (wt %) | Non-gelled | Gelled |
|---|---|---|
| 80 | 5.8 (±0.6) | 14.5 (±1.2) |
| 90 | 6.7 (±0.3) | 10.1 (±1.6) |

Thus, it is suggested that the gellant itself also has an influence on the structure of the delivery system, as once the gellant is added, the domains slightly grow in size and transform to an elongated shape, rather than assembling into globular droplets.

In order to characterize the structure of the oily domains, self-diffusion NMR (SD-NMR) analysis was carried out. SD-NMR analysis provides an indication on the location of each component within the structure, by calculating the diffusion coefficient of each component in the system. Rapid diffusion (>100×10⁻¹¹ m²s⁻¹) is characteristic of small or free molecules in solution, while slow diffusion coefficients (<0.1×10⁻¹¹ m²s⁻¹) suggest low mobility of macromolecules or bound/aggregated molecules.

SD-NMR measurements were performed with a Bruker AVII 500 spectrometer equipped with GREAT 1/10 gradients, a 5 mm BBO and a 5 mm BBI probe, both with a z-gradient coil and with a maximum gradient strength of 0.509 and 0.544 T m⁻¹, respectively. Diffusion was measured using an asymmetric bipolar longitudinal eddy-current delay (bpLED) experiment, or and asymmetric bipolar stimulated echo (known as one-shot) experiment with convection compensation and an asymmetry factor of 20%, ramping the strongest gradient from 2% to 95% of maximum strength in 32 steps. The spectrum was processed with the Bruker TOPSPIN software. NMR spectra were recorded at 25±0.2° C. The components were identified by their chemical shift in 1H NMR.

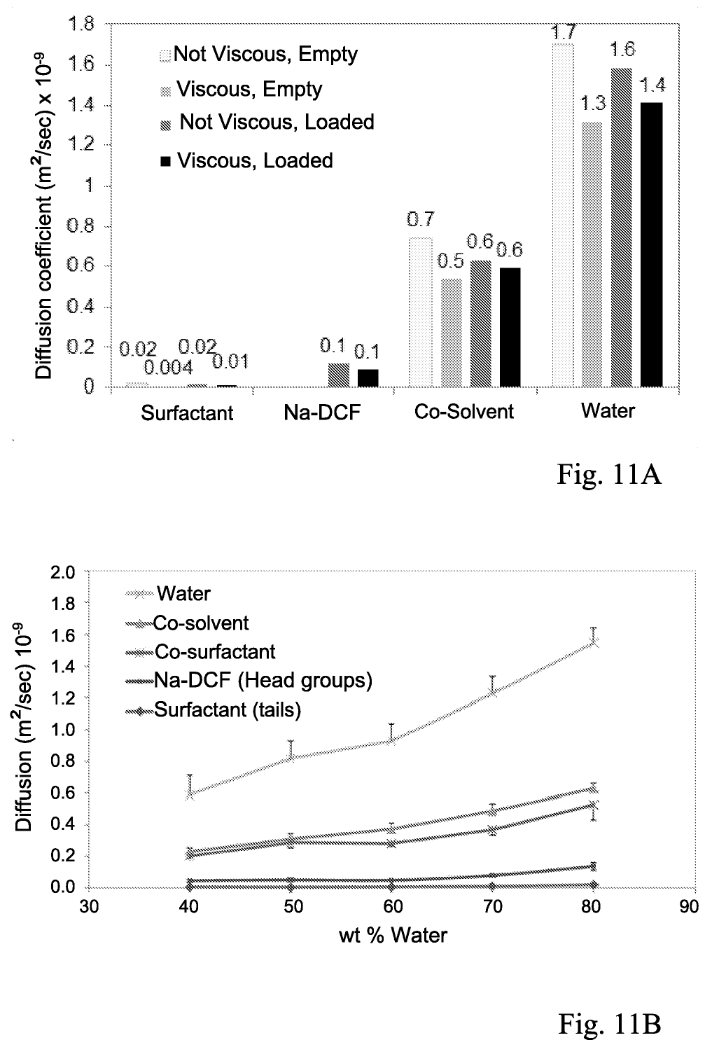

FIG. 11A shows the diffusion coefficients (Dx, m²/sec) of the main components for 2 wt % DCF-Na un-loaded and loaded formulation, at 80 wt % water, in non-viscosified (non-gelled) and viscosified (gelled) systems. FIG. 11B shows the effect of dilution on the diffusion coefficient of the loaded and gelled formulation.

As can be seen from FIGS. 11A-11B, the diffusion coefficient of DCF-Na is similar to that of the hydrophilic surfactants compared to the other components in the system. The Na-DCF diffuses slightly faster than the tails of the surfactants indicating that the Na-DCF is located at the interface and not within the oil core of the oily domains (as the formulation is very poor in oil). Further, the results indicate that the polar solvents are mostly located in the layer, far from the surfactants' heads, however still interact with the heads and are not entirely free (for surfactant tails Dx=0.02×10⁻¹¹ and for DCF-Na Dx=0.1×10⁻¹¹).

This suggests that binding occurs between DCF-Na and the surfactants' heads, suggesting that the DCF-Na molecules are interlocked by the surfactant's tails at the interface of the oily domains, and the DCF-Na molecules may also function as a co-surfactant.

It is also noted that the diffusion coefficient of DCF-Na is lower in the gelled formulation than in the non-viscosified system. Such reduction also contributes to the increased stability of DCF-Na in the viscosified/gelled system and provides for better control over the release of DCF-Na from the oily domains once applied onto the skin.

From the SD-NMR results, the so-called “obstruction factor (OF)” can be calculated. This factor is derived from the diffusivity of each component in the structure at each certain dilution point normalized to diffusion coefficient of the component itself in a liquid form or in a reference solution [OF=D/Do]. The obstruction factor is suggestive of the resistance of the components to be released from the structure at a given solubilizate concentration of DCF-Na (2 wt %). It can be seen that due to their close behavior and diffusion coefficients correlation, the components that are hindering the release of Na-DCF from the interface are the set of the surfactants. Low OF values of 0.1 to 0.2 are indicating of significant binding effects of the DCF-Na to the surfactants and, hence, slower release and the formation of a depot effect. The solvents and the water are not obstructing the drug molecule (OF values of 0.5 and 0.6).

### Gelled DCF-Na Formulation Compared to Commercial Product

Gelled DCF-Na formulations were prepared as described above. Their various properties were compared to Voltaren Emulgel®Forte, which is currently the leading commercial product for topical delivery of diclofenac. Voltaren Emulgel®Forte contains 2.32 wt % diclofenac diethylamine (DCF-DEA, which is comparable to 2 wt % DCF-Na) in a gelled emulsion formulation that primarily comprises inactive ingredients (excipients) such as butylhydroxytoluene, carbomers, cocoyl caprylocaprate, diethylamine, isopropyl alcohol, liquid paraffin, macrogol cetostearyl ether, oleyl alcohol, propylene glycol, and purified water.

### Visual Appearance

The physical properties of 2 wt % gelled DCF-Na formulation, at 80 wt % water dilution (named for ease of reference NDS 506(A)) in comparison to Voltaren Emulgel®Forte, are provided in Table 10.

TABLE 10
Comparison of physical properties

| Parameter | NDS 506(A) | Voltaren Emulgel ® |
|---|---|---|
| Transparency | Transparent | Opaque |
| Color | Clear to | White opaque |
| | slightly yellow | |
| Texture | Gel | Gel |
| Microscopy ^{a} | Uniform | Uniform |
| Turbidity (NTU) ^{b} | 80-100 | 1900-2500 |
| pH ^{c} | 7.1-7.5 | 7.9 |
| Droplet size (nm) ^{d} | 6.2 | N/A |
| Poly Dispersion | 0.4 | N/A |
| Index (PDI) ^{d} | | |

Microscopy analysis: Nikon Eclipse 80i, magnification ×10, polarized light

Turbidity evaluation: HI 83414 Turbidity and free/Total Chlorine Meter by HANNA instruments (using calibration curve samples). All samples were diluted ×11 with distilled water, shaking at 300 RPM for 1 hour at room temperature

pH Measurements: SevenEasy Metller Toledo

Drop size examination: Zeta sizer, nano sizer (nano-s), MALVERN Instrument

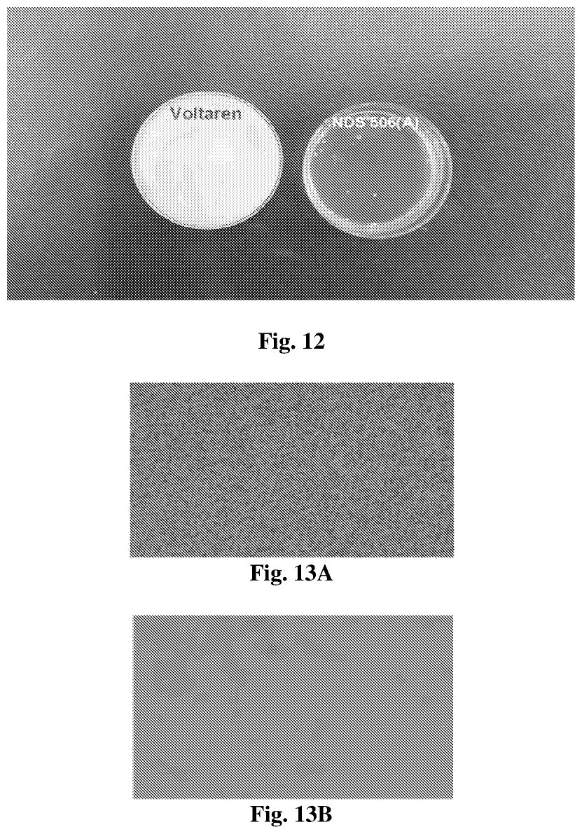

The differences in appearance between NDS 506(A) and Voltaren Emulgel@Forte are shown in FIG. 12, while microscopic images are provided in FIGS. 13A-13B.

Commercial products which are based on emulsions, such as Voltaren Emulgel® or Voltaren Emulgel@Forte, are typically a dispersion of two immiscible liquids, formed in the presence of emulsifiers/surfactants, which reduce the interfacial tension between the two phases and cover the dispersed droplets to retard aggregation, flocculation, coalescence and phase separation. Since the emulsifiers do not reduce the interfacial tension to zero and the coverage is not complete, emulsions require application of relatively high shear forces of multistage homogenizer to reduce the droplets size upon preparation of the emulsion. The resulting non-uniform droplets have a strong tendency to coalesce and/or result in phase separation, thereby stabilizing the system energetically. Thus, commercial product show a relatively non-uniform dispersity of the droplets together with large droplet size, far from being homogenous, resulting in a milky, white-opaque appearance.

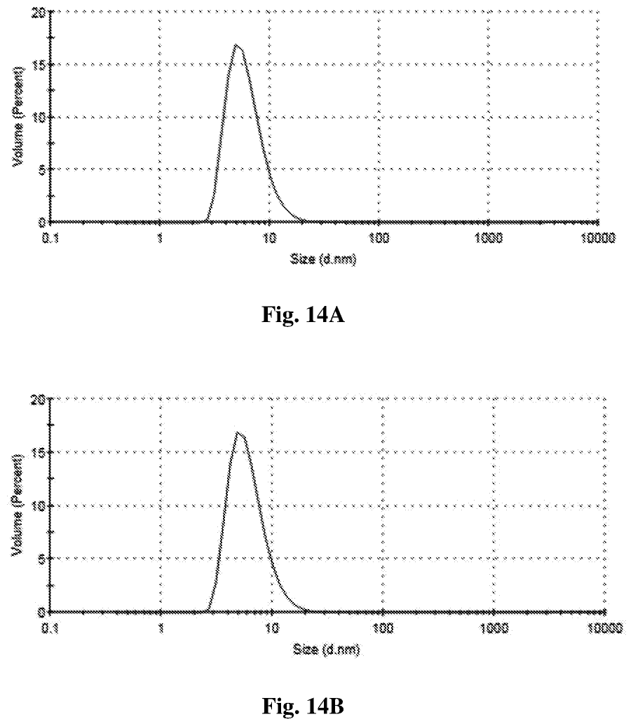

In comparison, the NDS 506(A) formulation are spontaneously formed as energetically balanced systems due to their substantially zero interfacial tension. Such formulations are characterized by a small and uniform oily domains size, as seen in FIGS. 14A-14B, resulting in transparent and stable systems.

### Viscosity and Rheology

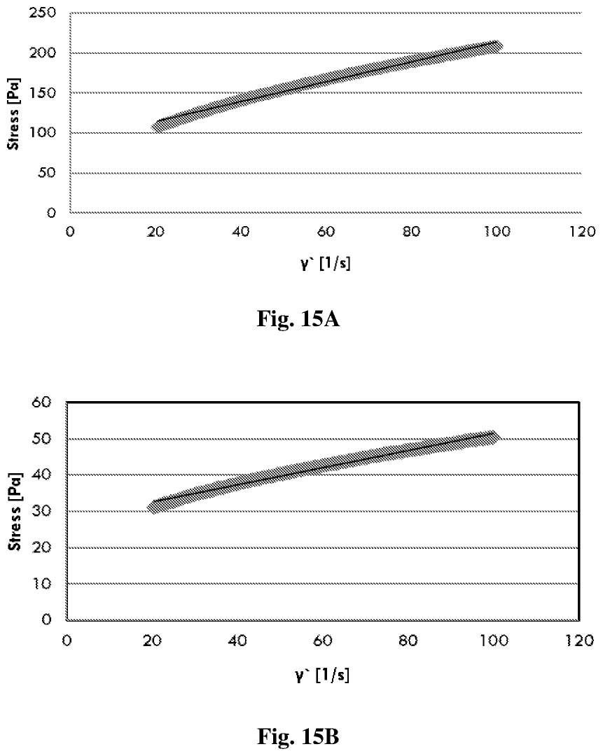

Rheological properties of Voltaren Emulgel® and NDS 506(A) was measured by ThermoHaake (Thermo Electron GmbH, Karlsruhe, Germany) using a cone (60 mm diameter) and glass plate, at 25±1° C., shear rates were 0-100s⁻¹, as shown in FIGS. 15A-15B, respectively.

As evident from the viscosity measurements, the viscosity of Voltaren Emulgel®Forte is significantly higher compared to that of NDS 506(A). As explained above, Voltaren Emulgel®Forte is a thermodynamically unstable emulsion, and hence requires relatively strong gelation and high viscosities in order to stabilize the emulsion. Further, such high viscosities often lower the absorbance of the formulation into the skin after application, and may also reduce the penetration and release of diclofenac into the skin and relevant tissues.

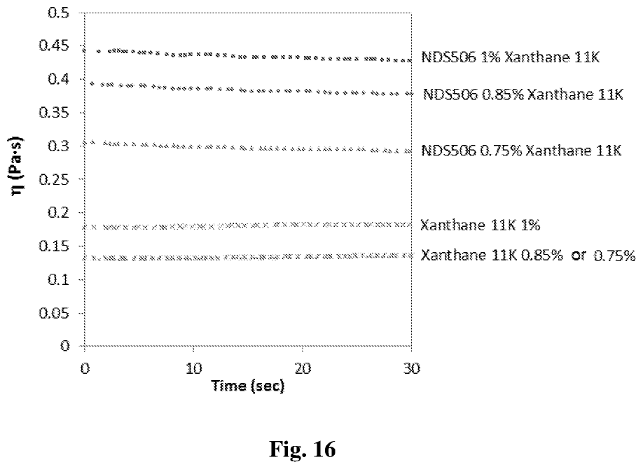

The viscosity of the gelled systems measured at 50 hz against time, demonstrated in FIG. 16 remains constant over time, and is generally dependent on the xanthan gum (or other viscosifying agent) concentration in the formulation.

As noted, the structures of empty systems are different than those formed by gelled systems loaded with DCF-Na. Since these differences were found to have significant effects on the release of DCF-Na from the delivery system, and hence on the formation of a depot effect, the rheological properties of each system was characterized.

Thus, the rheological properties of xanthan gel (i.e. the gelled aqueous phase, without the addition of the oily phase), the un-loaded gelled formulation and the DCF-Na loaded gelled formulation were measured and compared. The comparison provided data on the dynamic complex viscosity (l*), as well as the storage modulus (G′) and loss modulus (G″), which reflect the visco-elastic behavior of the systems.

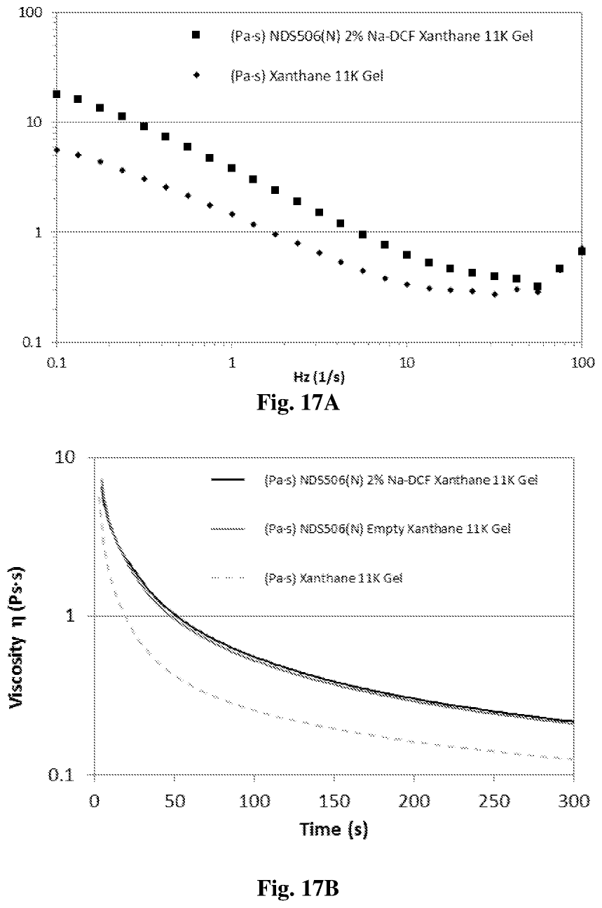

As seen in FIG. 17A, for both the gelled aqueous phase (without an oily phase) and gelled 2 wt % DCF-Na loaded formulation the complex viscosity drops significantly with the increase of shear rate, where at high shear rates the complex viscosity increases, indicating destruction of the gel structure (a gel-sol transition). However, it is important to note that the loaded gelled formulation shows higher complex viscosities throughout the shear rate sweep compared to the pure xanthan gel, indicating high stability of the formulation. As seen in FIG. 17B, the loading of DCF-Na into the gelled formulation has no significant effect on the viscosity, and its complex viscosity is similar to that of the un-loaded gelled system.

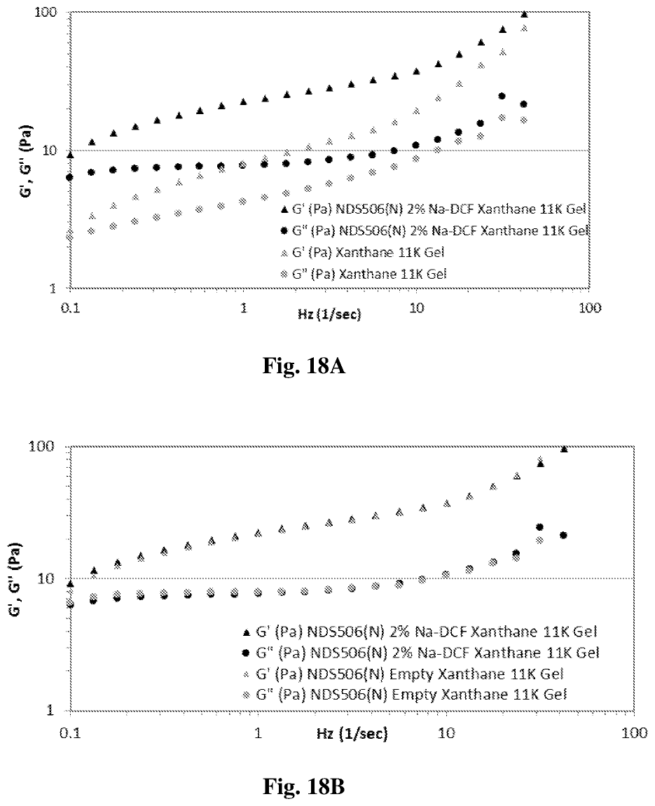

As seen in FIG. 18A, the storage and loss moduli (G′ and G″) of loaded gelled formulation are higher than that of the pure xanthan gel, meaning that the loaded gelled formulations have a higher energy storage. However, the loss of energy is smaller in the loaded gelled formulation compared to the pure xanthan gel, indicating that the loaded gelled formulation behaves in a viscoelastic manner, and is expected to form a viscoelastic film onto the skin once applied. From FIG. 18B it can be seen that the loading of DCF-Na into the gelled system has no effect on the storage and loss moduli.

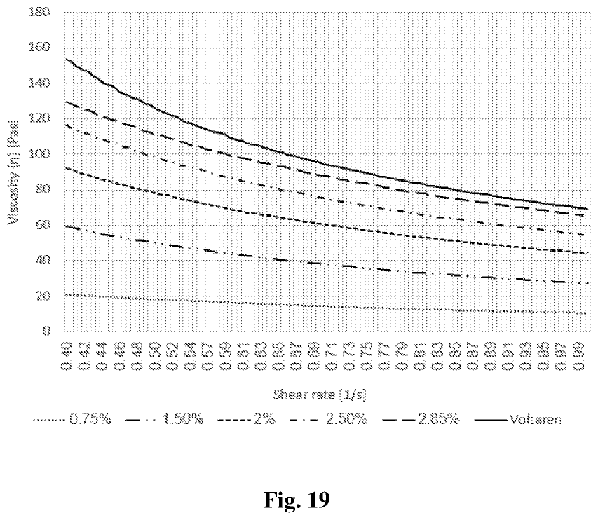

Further insight into the rheological characteristics of the formulations was investigated by measuring the complex viscosity at very low shear rates of the loaded systems with varying amounts of xanthan (0.75 wt % to 2.85 wt %) in comparison to Voltaren Emulgel®Forte (FIG. 19). Under these low shear rates, mimicking the rubbing of the gel onto the surface of the skin, the measured viscosity is lower compared to Voltaren Emulgel®Forte. However, with 2.85 wt % gellant, the formulation loss of viscosity against increasing shear rate drops slower and eventually is similar to the viscosity of the commercial emulsions (at 0.99 1/s). Without wishing to be bound by theory, the commercial emulsion has relatively large droplets and is highly anisotropic. Hence overcoming the interactions between the oil droplets in order to induce flow requires larger input of energy into the system (i.e. higher shear rates). The NDS 506(A) formulation, on the contrary, have smaller and homogenous nanodomains, resulting in a relatively isotropic system; these systems do not demonstrate significant interactions between the nanodomain, hence flow can be induced and maintained at very low shear rates.

As the formulations are designed for topical application, the viscosity of the formulations have an impact on their spreadability. This is demonstrated by utilizing a spreadability test.

Spreadability is assessed by placing 350 mg of a tested formulation in the middle of a clean, dry and uniform glass surface. The sample is covered by another glass surface having a weight of 180 g. After 60 second, the diameter of the spread sample is measured and compared to its initial diameter (before the weight was applied). The spreading value S is calculated by the following formula: S=m·A/t, in which m is the weight (g) placed on the sample, A is the spreading area (cm²) and t (sec) is the time the sample was exposed to the weight. Each formulations was tested 3 times.

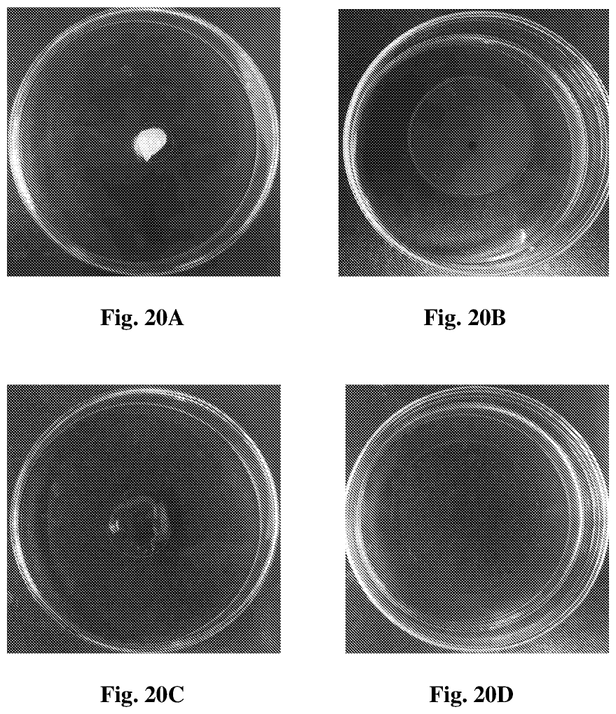

FIGS. 20A-20B show spreadability test for Voltaren Emulgel®Forte, while FIGS. 20C-20D show test results for NDS 506(A). As also seen from Table 11, formulation NDS 506(A) shows improved spreading compared to Voltaren Emulgel®Forte, indicating that NDS 506(A) can cover a larger skin surface using the given amount of formulation.

TABLE 11
Spreadability test results

| | | Mean | | |
|---|---|---|---|---|
| | Quantity | diameter | Mean area | Mean |
| Sample | (g) | (cm) | (cm²) | spreading |
| NDS 506(A) | 0.35 | 6.3 ± 0.1 | 31.17 ± 0.98 | 93.51 ± 2.96 |
| Voltaren | 0.35 | 4.1 ± 0.2 | 13.72 ± 1.28 | 39.67 ± 3.86 |
| Emulgel ®Forte | | | | |

### Sensorial Testing

NDS 506(A) was compared to Voltaren Emulgel®Forte in a series of sensorial tests. 20 human volunteers were asked to wash their hands thoroughly and completely dry them from any residues of water. A predefined weight amount of the formulation (350 mg of either NDS 506(A) or Voltaren Emulgel®Forte) material was placed on the back of their hand. The volunteers were asked to score the immediate contact feel of the gel in regards to its texture, consistency and creaminess using a scale of 1 to 6. Next, the volunteers were asked to rub-in the gel and score again from a scale of 1-6, the tackiness, greasiness and softness feel. In the last stage, the volunteers were asked to score the after-feel effect including softness, greasy, tackiness—residue and the possible performance of a film using the same scoring system as before.

As shown in Tables 12-1 and 12-2, various parameters were assessed before, during and after application onto the skin.

TABLE 12-1
Sensorial and textural test results for NDS 506(A) (score 1-6)

| Immediate contact | | Rub-in | | After feel | |
|---|---|---|---|---|---|
| Parameter | score | Parameter | score | Parameter | score |
| Texture | 6 | Tackiness | 0 | Soft | 6 |
| Consistency | 6 | Greasiness | 1 | Greasy | 0.5 |
| Creaminess | 4 | Softness | 6 | Tacky | 0.5 |
| — | — | Spreadability | 5 | Film residue | 0 |
| — | — | — | — | Absorbency | 6 |

TABLE 12-2
Sensorial and textural test results for Voltaren
Emulgel ®Forte (score 1-6)

| Immediate contact | | Rub-in | | After feel | |
|---|---|---|---|---|---|
| Parameter | score | Parameter | score | Parameter | score |
| Texture | 5 | Tackiness | 1 | Soft | 5 |
| Consistency | 3 | Greasiness | 1 | Greasy | 3 |
| Creaminess | 6 | Softness | 5 | Tacky | 2 |
| — | — | Spreadability | 5 | Film residue | 0.5 |
| — | — | — | — | Absorbency | 5 |

As evident from the sensory results, the NDS 506(A) formulation showed better sensorial and textural parameters, suggesting that such formulations are better absorbed into the skin. This may also contribute to improvement in user's compliance to treatment.

### Ex Vivo Permeation and Penetration of DCF-Na

Ex vivo permeability and penetration of NDS 506(A) was measured compared to Voltaren Emulgel®Forte using Franz cell diffusion (FC) system (PermeGear, Inc., Hellertown, PA), using freshly dermatome pig's ear skin. Comparison was carried out between NDS 506(A) and Voltaren Emulgel®Forte (2.32 wt % DCF-DEA). It is noted that 2.32 wt % DCF-DEA is comparable to 2.0 wt % DCF-Na.

Permeation Procedure Protocol: Five replicates of FC permeation studies were performed for each formulation sample. Skin samples selected showed no wounds, warts or hematomas. The skin's integrity was measured by Trans-epidermal water loss (TEWL) (Dermalab Cortex Technology instrument, Hadsund, Denmark). Only pieces showing TWEL levels less than 10±2.5 g/m² h were further used.

Skin was mounted on receiver chamber with stratum corneum (SC) facing upwards and the donor compartments were clamped in place. The receiver compartment was filled with freshly prepared phosphate buffer PBS (pH 7.4) with constant stirring using a Teflon-coated magnetic stirrer, while heated to 34±2° C. (depending on the RT) to produce 32° C. at the receptor cell. Before initializing the experiment the skin was left to acclimatize with pre-warmed (32° C.) 0.5 ml PBS placed in the donor cell.

After 30 minutes, PBS was removed and a defined infinite dosage (5 mg/cm²) of NDS 506(A) and Voltaren Emulgel®Forte were applied onto the skin by spreading the formulations homogeneously. The donor compartment was left open for 30 minutes to enable gel to adhere to the membrane properly and result in a fine film on the surface of the skin. Next, the donor cells and sampling port opening were sealed with Parafilm to avoid further evaporation.

0.5 ml samples were withdrawn from the receiver cell at predetermined intervals using a long glass Pasteur pipette to reach near the string area and achieve maximum homogeneity. Cells were replenished to their marked volume with fresh heated (32° C.) buffer solution. The addition of the solution to the receiver compartment was carried out with great care to avoid the entrapment of air bubbles beneath the dermis. Samples were taken to 1.5 ml amber vial and stored at −20° C. until analysis was completed.

All samples were measured using HPLC (Waters, Milford, MA/autosampler Waters 717 plus equipped with a photodiode array detector—Waters 996), according to the procedure described further herein. Diclofenac concentration of samples was evaluated from an eight point standards calibration curve, with a R² value not less than 0.999. Cumulative drug permeation (mcg/sq. cm) was calculated and plotted against time.

HPLC Waters 600 series; Autosampler Waters 717 plus; photodiode array detector Waters 996. Mobile phase: 65% acetonitrile/35% water/0.1% trifluoric-acetic acid or formic-acid. Column type: aqua 5 μm, C18, 250 mm×4.6 mm (Phenomenex). Guard column: SecurityGuard cartridge, C18, 4×3.0 mm ID (Phenomenex). Flow rate: 1 ml/min; 30° C.; injection volume 5 μl.

Penetration Procedure Protocol (Tape Striping) [9]: The procedure was followed as listed above. However, sampling from receptor cell was carried only after 24 hours. Remaining formulations were carefully removed from the donor cell using a spatula. The formulations were placed in a vial containing 10 ml methanol, and donor compartment was thoroughly washed, using the same methanol volume, to ensure that all residual formulation left on the glass was also removed.

An adhesive film was applied onto the skin surface and pressed using a constant weight roller to avoid the formation of furrows and wrinkles and enable a uniform adhesion of the tape. An additional strip was taken from the skin (total 3). Strips 1-3 were placed into the same vial together with the formulation. This vial content represents the diclofenac remaining in the donor sample (the formulation) after 24 hours together with that found on the surface of the skin termed “Formulation+Upper” (data not shown).

Seven additional strips (4-10 #) were pulled and placed together in a separate 10 ml methanol vial for the analysis of diclofenac depot skin effect termed “Deep skin”.

The remaining striped skin was place in a third 10 ml methanol vial for analysis of diclofenac content in the this skin layer, termed “Stripped Skin”). As a positive control, and to determined diclofenac content, the same amount of fresh formulation was dissolved in 10 ml methanol vial and recovery of all collected diclofenac concentration (from all steps) were combined form all layer and permeation to show ca. 90-100%.

All vials (except the samples taken from the receptor cell) were shaken at room temperature for 1.5 hr at 200 rpm and sonicated for 15 min. Samples were filtered using a 0.45 m cone and transferred into a clean new amber glass vials. All samples were measured using HPLC (same as above). Quantification of diclofenac was calculated from an eight standards calibration curve having a R² not less than 0.999.

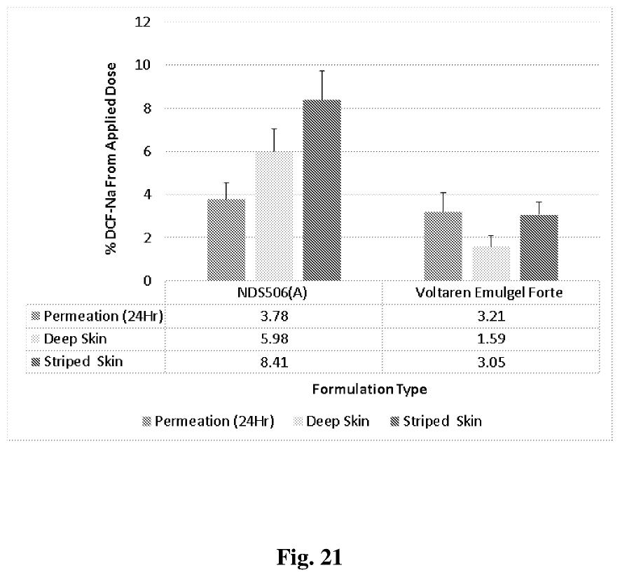

Comparative results of ex vivo tests are provided in FIG. 21. Measurements of the levels of DCF-Na within the skin layers (‘Deep’ and ‘Stripped Skin’) demonstrated an increased concentrations of the DCF-Na when testing formulation NDS506(A) compared to commercial Voltaren Emulgel® Forte (2.32 wt % diethylamine diclofenac). However, the permeation levels of the drug as measured after 24 hours in the receiver cell were similar within all three tested formulations. This demonstrates that the permeation of DCF-Na using NDS506(A) reaches deeper skin levels in higher concentrations and then to the desired tissue compared to the reference product, with limited systemic exposure. Without wishing to be bound to theory, the Franz cell analysis results demonstrates the skin depot effect of NDS506(A) and its permeation to the applied joint treated tissue with limited systemic exposure.

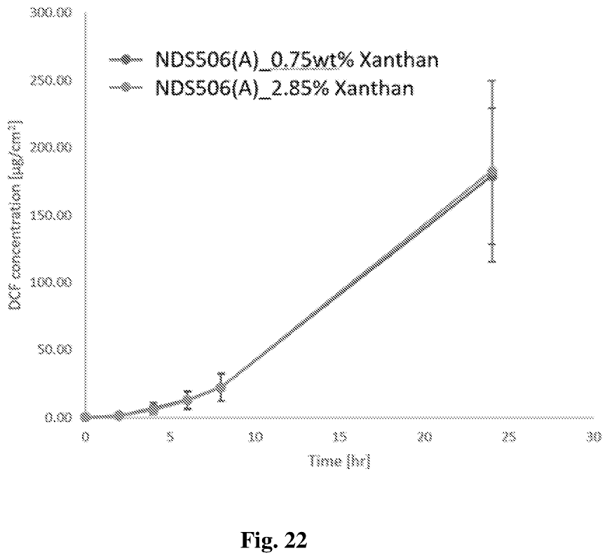

Increasing the xanthan content from 0.75 wt % to 2.85 wt % did not have an effect on the permeation of DCF-Na in a Franz cell test, as seen in FIG. 22. Namely, although the viscosity of the formulation was increased and a denser or stronger network was formed in the aqueous phase, this did not hinder the release of DCF-Na from the formulation.

### Stability

The stability of NDS 506(A) with antioxidants was evaluated for a period of 3 months, at four different temperatures and relative humidity (% RH) conditions (4° C., 25° C./60% RH and 40° C./75% RH).

Appearance, pH, % DCF-Na (by HPLC) were measured at each time point for triplicate samples, and compared to the initial measurements taken immediately after preparation of the formulations. The results are presented in Table 13.

NDS506(A) was also found to maintain stability through 72 hours of freezing and thawing back to room temperature (data not shown), namely the formulation's structure was maintained, without any phase separation or changes in the appearance of the formulation.

TABLE 13
3 months stability

| | | | 25° C. | 40° C. |
|---|---|---|---|---|
| Test conditions | Initial | 4° C. | 60% RHA | 75% RHA |
| Incubation time | — | 3 months | 3 months | 3 months |
| Appearance | Homogenous, | Homogenous, | Homogenous, | Homogenous, |
| | transparent | transparent | transparent | transparent |
| | slightly- | slightly- | slightly- | slightly- |
| | yellow weak | yellow weak | yellow weak | yellow weak |
| | gel | gel | gel | gel |
| pH | 7.25 | 7.32 | 7.33 | 7.21 |
| Assay (avg. label | 101.21 ± 0.91 | 98.35 ± 1.35 | 99.35 ± 1.35 | 100.04 ± 1.77 |
| claim % ± % RDS) | | | | |

As can be observed, the DCF-Na loaded gelled formulations maintain their clarity, pH and active concentration values over prolonged periods of time, i.e. at least up to 3 months, when stored at various storage conditions. Thus, these formulations may be stored for prolonged periods of time without adversely affecting their properties.

To determine long term stability of formulations, a rapid measurement was carried out using LUMiFuge™ analytical centrifugation. LUMiFuge™ analysis enables to predict the shelf-life of a formulation in its original concentration, even in cases of slow destabilization processes like creaming, sedimentation, flocculation, coalescence and fractionation. During LUMiFuge™ measurements, parallel light illuminates the entire sample cell in a centrifugal field; the transmitted light is detected by sensors arranged linearly along the total length of the sample-cell. Local alterations of particles or droplets are detected due to changes in light transmission over time. The results are presented in a graph plotting the percentage of transmitted light (Transmission %) as a function of local position (mm), revealing the corresponding transmission profile over time.

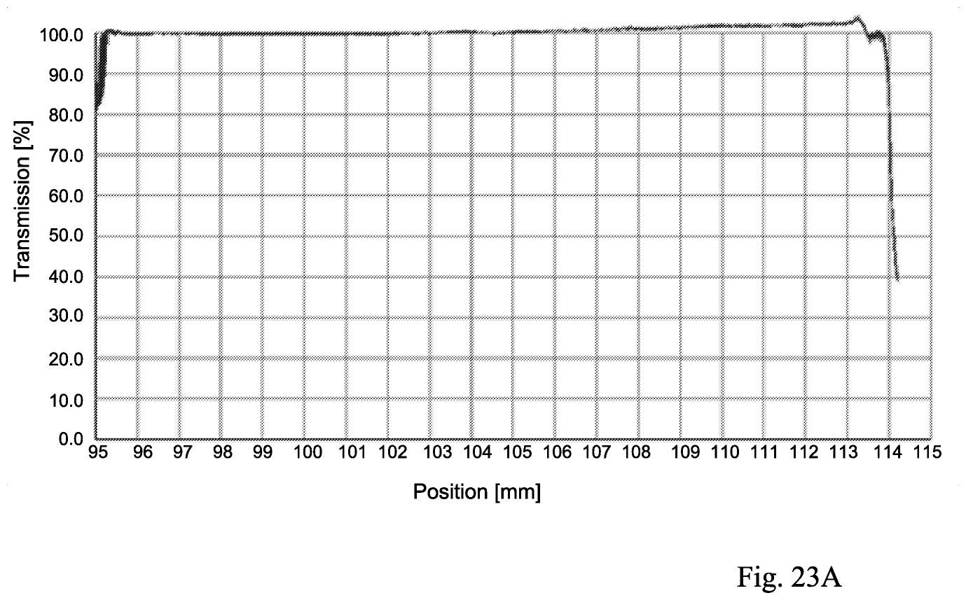

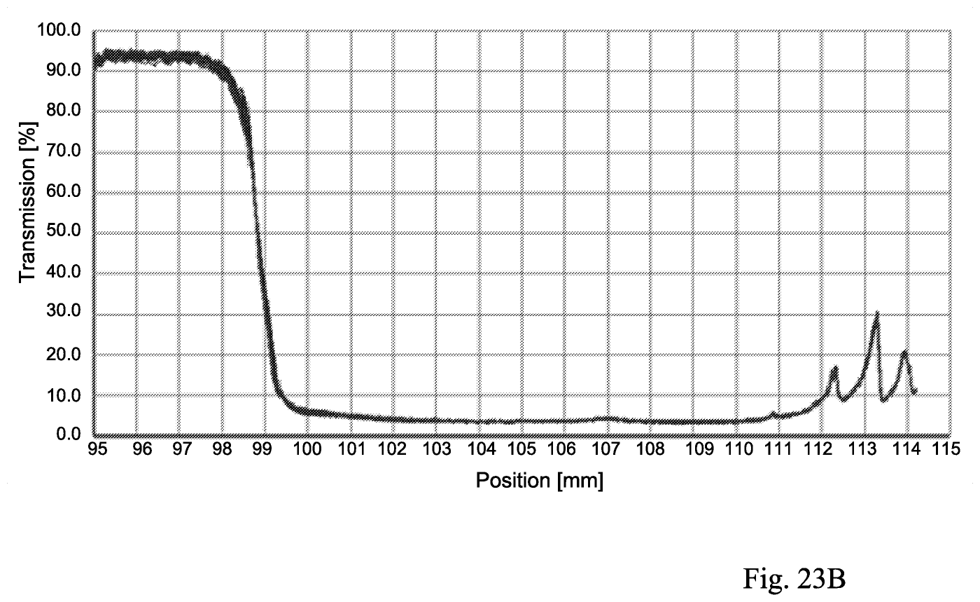

LUMiFuge™ test results for NDS 506(A) and typical commercial emulsion are shown in FIGS. 23A-23B, respectively, over a time period of 24 hours.

The analysis of emulsion (having white milky appearance) scattered and absorbed the light resulting in significant decrease in light transmission over time, as the gelled emulsion's stability was impaired. In contrast, the NDS 506(A) formulations, (having a clear and transparent appearance) enabled light to be transmitted (100%) throughout the whole measured cell length. The transmitted light, reflecting the transparency of the sample, was even obtained over 24 hours of centrifugal forces of 3000 rpm tested during analysis. These results support expectation for long shelf life stability properties of the NDS 506(A) formulation after long storage conditions.

### Stability to Freezing and Thawing

Stability to freezing and thawing was assessed by placing a sample of formulation NDS506(A) at −20° C. for 72 hours and then thawing at room temperature for 2 hours. The formulations remained clear and homogenous after freezing and thawing, with no apparent change.

1.-79. (canceled)

80. A topical formulation comprising an oily phase integrated into a gelled aqueous continuous phase, the oily phase being in the form of oily nano-domains dispersed in the continuous phase,
wherein the oily phase comprises an active agent, at least one oil, at least two hydrophilic surfactants, at least two polar solvents, and at least two penetrating promotors, and
wherein the gelled aqueous continuous phase comprises an aqueous diluent and at least one gellant; and
said at least two hydrophilic surfactants comprise a first hydrophilic surfactant and a second hydrophilic surfactant;
wherein said first and second hydrophilic surfactants are each selected from the group consisting of polyoxyethylene sorbitan monolaurate, polyoxyethylene sorbitan monopalmitate, polyoxyethylene sorbitan monooleate, polyoxyethylene sorbitan monostearate, polyoxyethylene esters of saturated castor oil, polyoxyethylene esters of unsaturated castor oil, ethoxylated monoglycerol esters, ethoxylated sorbitan monooleate/linoleate, ethoxylated sorbitan trioleate, ethoxylated castor oil, hydrogenated castor oil, ethoxylated hydrogenated castor oil, hydroxystearate, ethoxylated fatty acids, ethoxylated alcohols of fatty acids, oleoyl macrogolglycerides, polyglycerol esters of fatty acids, ethoxylated mono glycerides, and ethoxylated diglycerides;
provided that the first hydrophilic surfactant is different from the second hydrophilic surfactant,
said at least two polar solvents comprising at least a first polar solvent selected from short chain alcohols, and at least a second polar solvent selected from polyols,
the formulation comprises between about 0.5 wt % and about 3 wt % of said at least one oil,
the formulation comprises between about 2 wt % ad about 10 wt % of said penetrating promotors, and
said active agent being diclofenac or any pharmaceutically acceptable salt thereof.

81. The topical formulation of claim 80, wherein the oily phase further comprises at least one lipophilic co-surfactant.

82. The topical formulation of claim 81, wherein the lipophilic co-surfactant is a phospholipid.

83. The topical formulation of claim 80, wherein the oily domains have an average domain size of between 5 and 150 nm.

84. The topical formulation of claim 80, wherein the oily domains have an aspect ratio of between about 1.1 and 1.5.

85. The topical formulation of claim 80, wherein the oily domains have an elongated shape.

86. The topical formulation of claim 80, wherein the active agent is selected from the group consisting of diclofenac, diclofenac sodium (DCF-Na), diclofenac potassium (DCF-K), DCF-ammonium, diclofenac diethylamine (DCF-DEA) and mixtures thereof.

87. The topical formulation of claim 86, wherein said active agent is present in the formulation at an amount of between about 1 wt % and about 6 wt %.

88. The topical formulation of claim 80, wherein said oil is selected from the group consisting of isopropyl-myristate (IPM), ethyl oleate, methyl oleate, lauryl lactate, oleyl lactate, oleic acid, linoleic acid, monoglyceride oleate and monoglyceride linoleate, coco caprylocaprate, hexyl laurate, oleyl amine, oleyl alcohol, hexane, heptanes, nonane, decane, dodecane, short chain paraffinic compounds, terpenes, D-limonene, L-limonene, DL-limonene, olive oil, soybean oil, canola oil, cotton oil, palmolein, sunflower oil, corn oil, essential oils, peppermint oil, pine oil, tangerine oil, lemon oil, lime oil, orange oil, citrus oil, neem oil, lavender oil, anise oil, pomegranate seed oil, grapeseed oils, pumpkin oil, rose oil, clove oil, sage oil, eucalyptol oil, jasmine oil, oregano oil, capsaicin, triglycerides, unsaturated tocopherols, polyunsaturated tocopherols, medium-chain triglycerides (MCT), avocado oil, punicic acid, conjugated linoleic acid (CLA) fatty acids, omega 3-fatty acids, 6-fatty acids, 9-fatty acids, ethyl esters of omega fatty acids, and mixtures thereof.

89. The topical formulation of claim 80, wherein the oil is selected from the group consisting of isopropyl-myristate (IPM), oleic acid, oleyl alcohol, vegetable oils, terpenes, peppermint oil, eucalyptol oil, and mixtures thereof.

90. The topical formulation of claim 80, wherein said first and second hydrophilic surfactants are each being independently selected from the group consisting of ethoxylated sorbitan monolaurate, ethoxylated sorbitan monostearate/palmitate, ethoxylated sorbitan monooleate/linoleate, ethoxylated sorbitan trioleate, ethoxylated castor oil, hydrogenated ethoxylated castor oil, ethoxylated monoglyceride stearate/palmitate, polyoxyl 40 castor oil, polyoxyl 35 castor oil, polysorbate 20, polysorbate 40, polysorbate 60, polysorbate 80, polyoxyethylene-40-stearate, polyoxyethylene-20-stearate, oleoyl macrogolglycerides, polyglyceryl-3 dioleate, ethoxylated hydroxyl stearic acid, and polyglycerol esters of fatty acids.

91. The topical formulation of claim 80, wherein said at least two penetrating promotors are independently selected from the group consisting of dimethyl sulfoxide (DMSO), dimethyl isosorbide (DMI), isopropyl myristate (IPM), 2-(2-ethoxyethoxy)ethanol, phosphatidylcholine (PC), ethanol, isopropyl alcohol (IPA), ethyl acetate, oleyl alcohol, oleic acid, oleyl esters, beta-cyclodextrines, urea, dimethyl urea, diphenyl urea, glycerol and propylene glycol (PG), pyrrolidone, peppermint oil, terpene, terpenoids oils, essential oils and combinations thereof.

92. The topical formulation of claim 80, wherein the total amount of the first and second hydrophilic surfactants in the formulation is between about 2 wt % and about 12 wt %.

93. The topical formulation of claim 80, wherein said first hydrophilic surfactant is present in the formulation in an amount of between about 1.75 wt % and about 8.0 wt % and said second hydrophilic surfactant is present in the formulation in an amount of between about 0.45 wt % and about 3.8 wt %.

94. The topical formulation of claim 80, wherein the total amount of the first and second solvents in the formulation is between about 2.5 wt % and about 25 wt %.

95. The topical formulation of claim 80, wherein the first hydrophilic surfactant is selected from polysorbate 40, polysorbate 60, polysorbate 80, oleoyl macrogolglycerides, polyglyceryl-3 dioleate, or ethoxylated hydroxyl stearic acid, and the second surfactant is selected from ethoxylated castor oil, hydrogenated castor oil or ethoxylated hydrogenated castor oil.

96. The topical formulation of claim 80, wherein the first hydrophilic surfactant is polysorbate 60 and the second surfactant is selected from ethoxylated castor oil or hydrogenated castor oil.

97. The topical formulation of claim 80, wherein the first hydrophilic surfactant is selected from ethoxylated fatty alcohols of short, medium or long chain fatty acids.

98. The topical formulation of claim 80, wherein each of the first and second hydrophilic surfactants is independently selected from ethoxylated fatty alcohol, polyoxyethylene sorbitan monostearate or decaglycerol monooleate, provided that the first hydrophilic surfactant is different from the second hydrophilic surfactant.

99. The topical formulation of claim 80, wherein said first hydrophilic surfactant is selected from the group consisting of polyoxyethylene sorbitan monolaurate, polyoxyethylene sorbitan monopalmitate, polyoxyethylene sorbitan monooleate, and polyoxyethylene sorbitan monostearate, and said second hydrophilic surfactant is selected from polyoxyethylene esters of saturated castor oil, polyoxyethylene esters of unsaturated castor oil, ethoxylated castor oil, ethoxylated hydrogenated castor oil, ethoxylated fatty acids or ethoxylated fatty alcohols.
